(19) United States
(12) Patent Application Publication
(10) Pub. No.: US 2007/0156225 A1
(43) Pub. Date: Jul. 5, 2007

(54) AUTOMATED CONTROL MECHANISMS AND METHODS FOR CUSTOM LENGTH STENT APPARATUS

(75) Inventors: Robert George, San Jose, CA (US); Bernard Andreas, Redwood City, CA (US)

Correspondence Address: TOWNSEND AND TOWNSEND AND CREW, LLP;(CLIENT NO 021629-000000), TWO EMBARCADERO CENTER, 8TH FLOOR, SAN FRANCISCO, CA, 94111-3834, US

(73) Assignee: Xtent, Inc., Menlo Park, CA (US)

(21) Appl. No.: 11/614,271

(22) Filed: Dec. 21, 2006

Related U.S. Application Data
(63) Continuation-in-part of application No. 10/746,466, filed on Dec. 23, 2003.

(51) Int. Cl. A61F 2/84

(52) U.S. Cl. 623/1.12; 623/1.11; 606/108

(57) ABSTRACT

Methods and apparatus for delivering prosthetic segments to a body lumen, utilize a delivery device having an elongated flexible member including proximal and distal ends, a plurality of prosthetic segments arranged near the distal end and axially along the elongated flexible member and an outer sheath slidably disposed over at least a portion of the prosthetic segments. The delivery device also includes a control mechanism coupled with the outer sheath and the elongated flexible member, wherein the control mechanism is adapted to retract the sheath a fixed distance, the fixed distance selectable by an operator to expose a selected number of prosthetic segments and create a spacing between a proximal prosthetic segment in the selected number and a distal prosthetic segment remaining with the elongated flexible member.

### CROSS-REFERENCES TO RELATED APPLICATIONS

The present application is a continuation-in-part of U.S. patent application Ser. No. 10/746,466 (Attorney Docket No. 021629-002200US), filed Dec. 23, 2003, the full disclosure of which is hereby incorporated by reference.

### BACKGROUND OF THE INVENTION

1. Field of the Invention.

This invention relates generally to medical apparatus and methods, and more specifically to vascular catheters, stents and stent delivery systems for use in the coronary arteries and other vessels.

Stenting is an important treatment option for patients with coronary artery disease. The stenting procedure involves placing a tubular prosthesis at the site of a lesion, typically a stenotic region within a diseased coronary artery. The procedure is performed in order to maintain the patency of the artery and is often performed after a primary treatment such as angioplasty. Early stent results suffered from high rates of restenosis, i.e. the tendency for the stented coronary artery to become re-occluded following implantation of the stent. However, in recent years, restenosis rates have decreased substantially, due in part to drug eluting stents as well as other improvements in stent delivery methods and stent technology. As a result, the number of stent related procedures being performed worldwide continues to dramatically increase.

Stents are typically either self-expanding or balloon expandable and they are delivered to the coronary arteries using long, flexible vascular catheters typically inserted percutaneously through the patient's femoral artery. For self-expanding stents, the stent is simply released from the delivery catheter and it resiliently expands into engagement with the vessel wall. For balloon expandable stents, a balloon on the delivery catheter is expanded which in turn expands and deforms the stent to the desired diameter, whereupon the balloon is deflated and removed, leaving the stent in place.

Current stent delivery technology suffers from a number of drawbacks which can make delivery of stents challenging. In particular, current stent delivery catheters often employ stents having fixed lengths. The proper selection of fixed length stents requires accurate knowledge of the lesion length being treated. While lesion length may be measured prior to stent deployment using angiography and fluoroscopy, these measurements are often inaccurate. Thus, if an incorrectly sized stent is introduced to a treatment site, it must be removed from the patient along with the delivery catheter and replaced with a different device having the correct stent size. This prolongs the procedure, increases waste and results in a more costly procedure.

The use of “custom length” stents as an alternative to fixed length stents has been proposed. One such approach for providing a custom length stent has been to use segmented stents for treatment in which only some of the stents are deployed for treatment. Several exemplary systems are described in several copending, commonly assigned applications which are listed below. In these systems, the stent segments are deployed by selective advancement over the delivery catheter. After delivering an initial group of segments, the catheter may be repositioned to a new treatment site and a further group of segments can then be deployed. These systems enable treatment of multiple lesions with a single device and may contain up to fifty segments. While this technology represents a significant improvement over earlier stent delivery systems, in the case of smaller, more focal lesions or single lesions, only a small number of stent segments are needed and thus there is considerable waste when a large number of stent segments remain undeployed and end up being discarded at the end of the procedure.

Another challenge with existing “custom length” stent delivery systems is that to deliver multiple stent segments to multiple lesion sites requires an intricate delivery system that can be somewhat complex to use. Thus, a simpler delivery system that can be operated with one hand and allows length customization with fewer prosthetic segments on the delivery catheter is desirable, especially for use in treating a single lesion.

For the above reasons as well as others, it would be desirable to provide improved prosthetic stents and delivery catheters. It would be particularly desirable to provide catheters which enable stent length to be customized yet have a minimal quantity of stent segments so as to treat common lesion lengths while minimizing stent segment waste. It is also desirable to provide a delivery system that is flexible and can track torturous vessels and that has a simple construction and is easy to use in deploying a selectable number of stent segments to a single treatment site.

2. Description of the Background Art.

Prior publications describing catheters for delivering multiple segmented stents include: U.S. Publication Nos. 2004/0098081, 2005/0149159, 2004/0093061, 2005/0010276, 2005/0038505, 2004/0186551 and 2003/013266. Prior related unpublished co-pending U.S. patent applications include Ser. No. 11/148,713, filed Jun. 8, 2005 (Attorney Docket No. 14592.4002), entitled “Devices and Methods for Operating and Controlling Interventional Apparatus”; Ser. No. 11/148,545, filed Jun. 8, 2005 (Attorney Docket No. 14592.4005), entitled “Apparatus and Methods for Deployment of Multiple Custom-Length Prosthesis”; Ser. No. 11/344,464, filed Jan. 30, 2006 (Attorney Docket No. 021629-003500US), entitled “Apparatus and Methods for Deployment of Custom-Length Prostheses”; Ser. No. 60/784,309, filed Mar. 20, 2006 (Attorney Docket No. 021629-003600US), entitled “Apparatus and Methods for Deployment of Linked Prosthetic Segments”; Ser. No. 11/469,773 filed Sep. 1, 2006 (Attorney Docket No. 021629-004000US), entitled “Custom Length Stent Apparatus”; and Ser. No. 11/462,951 filed Aug. 7, 2006 (Attorney Docket No. 021629-004100US), entitled “Custom Length Stent Apparatus.” The full disclosures of each of these patents and applications are incorporated herein by reference.

### BRIEF SUMMARY OF THE INVENTION

The invention generally provides for the delivery of prosthetic segments with a flexible delivery catheter capable of navigating torturous vessels such as the coronary arteries. The delivery catheter permits deployment of a selectable number of prosthetic segments at a single treatment site, thus allowing customization of prosthesis length while the delivery catheter is in a body lumen at a treatment site. Customization of prosthesis length in situ permits better matching of the prosthesis length to the lesion length being treated. The delivery catheter has a simplified design with a control mechanism on the catheter handle for selecting prosthetic segments for deployment.

The terms “stent” and “stenting” are defined to include any of the array of expandable prostheses and scaffolds which are introduced into a lumen at a target treatment site and expanded in situ thereby exerting a radially outward force against the lumen wall. The prostheses of the present invention comprise a closed or an open lattice structure and are typically fabricated from a malleable or elastic material. When a malleable material is used, such as stainless steel, gold, platinum, titanium, cobalt chromium and other alloys, the stents are typically expanded by balloon inflation, causing plastic deformation of the lattice so that it remains permanently deformed in the open position after deployment. When formed from an elastic material, including superelastic materials such as nickel-titanium alloys, the lattice structures are commonly constrained radially during delivery and upon deployment the constraining structure is removed, allowing the prosthesis to “self-expand” at the target site. The terms “stent,” “prosthetic segment” and “stent segments” refer broadly to all radially expansible stents, grafts, and other scaffold-like structures which are intended for deployment within a body lumen.

In a first aspect of the present invention, an apparatus for delivering prostheses in a body lumen comprises an elongated flexible member having a proximal end, a distal end and a plurality of radially-expandable prostheses in an unexpanded condition arranged axially along the elongated flexible member near the distal end. The apparatus also comprises an outer sheath slidably disposed over at least a portion of the prostheses and a control mechanism coupled with the outer sheath and the elongated flexible member. The control mechanism includes means for selecting a plurality of prostheses to deploy in a range from 2 to 20, and the control mechanism also retracts the sheath a fixed distance to expose the selected plurality of prostheses outside the sheath in the unexpanded condition.

Often the control mechanism is adapted to create a space free of the prostheses between a proximal prosthesis in the selected plurality and a distal end of the sheath with the prostheses in the unexpanded condition. The control mechanism may create the space with a predetermined length and often the control mechanism is adapted to permit operator adjustment of the length of the space after creating the space. The control mechanism often is held by an operator using one hand while simultaneously being actuated with one or more fingers or a thumb of the one hand.

The apparatus may also comprise a handle that is coupled to the proximal end of the elongated flexible member. Usually, actuation of the control mechanism retracts the sheath a fixed distance equal to a predetermined multiple of the length of each prosthesis. The control mechanism usually comprises an actuator that is movably coupled to the handle. The handle usually is held by an operator using one hand while simultaneously actuating the actuator with one or more fingers or a thumb of one hand. The control mechanism often has an axially movable slider disposed on the handle, a pair of triggers pivotably coupled to the handle, or a lever. Either the actuator or a control mechanism having an actuator, is movable through a fixed distance, and each actuation of the actuator retracts the outer sheath a distance equal to the length of one prosthetic segment or a predetermined multiple thereof, and sometimes this includes the predetermined length.

The control mechanism may comprise a selector switch disposed on the handle, and the selector switch has a plurality of positions corresponding to different numbers of the prostheses to be included in the selected plurality. The control mechanism may also include a movable actuator, and movement of the actuator retracts the sheath a distance which varies according to the position of the selector switch. The actuator is often movable through a distance which varies according to the position of the selector switch. The actuator may also be movable through a fixed distance and moving retracts the sheath a distance equal to the length of one of the prostheses or a predetermined multiple thereof.

The control mechanism often includes a ratchet mechanism, a rotationally or linearly movable mechanism or a plurality of axially movable sliders on the handle, with the sliders being movable through a fixed distance. A first of the sliders is usually movable through a first fixed distance and a second of the sliders is movable through a second fixed distance different than the first distance. Often, when the first slider is moved through the first fixed distance the selected plurality of prostheses exposed outside the sheath is a different number than when the second slider is moved through the second fixed distance. The control mechanism is also often adapted to provide audible, visual or tactile feedback during actuation of the control mechanism so as to indicate the number of prostheses in the selected plurality.

The control mechanism may comprise a first control adapted to expose the selected number of prostheses and a second control adapted to create the spacing. The second control often is an actuator movably coupled to the handle and the handle may be held by an operator using one hand while simultaneously actuating the second control with one or more fingers or a thumb of the one hand. The second control may include an axially movable slider disposed on the handle or a lever pivotably coupled to the handle. The second control is usually rotationally or linearly movable. The second control also may be movable through a predetermined distance and moving the second control creates the spacing with a predetermined length. The second control is not usually movable beyond the predetermined distance, and usually provides feedback to the operator when moved through the predetermined distance. The feedback may comprise a visual indicator associated with the second control or a detent engaged by the second control. Often, after the moving through the fixed distance the second control is further movable to adjust the length of the spacing.

The apparatus may further comprise a pusher element disposed on the elongated flexible member that prevents the prostheses from axially moving toward the proximal end of the elongated flexible member. The apparatus may also comprise an engaging member disposed near a distal end of the outer sheath that can engage one or more of the prostheses so as to facilitate movement of the prostheses axially on the elongated flexible member. The apparatus may also comprise an expandable member such as a balloon coupled to the flexible member near the distal end, and the prosthetic segments are positionable on the balloon for expansion therewith. The sheath is often adapted to constrain the expandable member from expansion when positioned thereover. Upon retraction of the sheath a selected portion of the expandable member is usually exposed for expansion of the selected plurality of prostheses.

The apparatus may also comprise a switch having a first position and a second position. Upon actuation of the control mechanism in the first position, the prostheses are exposed and when the switch is in the second position the spacing in created. The apparatus may include an indicator adapted to display the selected number of prostheses exposed from the sheath. Sometimes the control mechanism comprises a length selector having a plurality of settings, with each setting corresponding to the number of prostheses to be exposed. Upon actuation the sheath is retracted a distance to expose the number of prostheses corresponding to a selected setting.

In another aspect of the present invention, an apparatus for delivering prostheses in a body lumen comprises an elongated flexible member having a proximal end, a distal end and a plurality of prostheses arranged axially along the elongated flexible member near the distal end. An outer sheath is slidably disposed over at least a portion of the prostheses and a control mechanism coupled with the outer sheath and the elongated flexible member has a first actuator which retracts the outer sheath a distance for exposing a selected number of prostheses. The control mechanism also has a second actuator which creates a space free of the prostheses between a proximal prosthesis in the selected number of prostheses and a distal end of the outer sheath. The control mechanism usually creates the space with a predetermined length and the control mechanism also permits operator adjustment of the length of the space after creating the space with the predetermined length.

Often, actuation of the control mechanism retracts the sheath a fixed distance equal to a predetermined multiple of the length of each prosthesis. The first actuator is usually movable through a fixed distance and each actuation of the first actuator retracts the outer sheath a distance equal to the length of one prosthetic segment or a predetermined multiple thereof. Sometimes the control mechanism comprises a plurality of axially movable sliders which are movable through a fixed distance. Sometimes, a first of the sliders is movable through a first fixed distance and a second of the sliders is movable through a second fixed distance different than the first fixed distance. When the first slider is moved through the first fixed distance the selected number of prostheses exposed outside the sheath is a different number than when the second slider is moved through the second fixed distance.

The apparatus may further comprise a handle coupled to the proximal end of the elongated flexible member. Often the handle is held by an operator using one hand while simultaneously actuating the first or second actuator with the fingers or thumb of the one hand. The first actuator may comprise an axially movable slider, a pair of triggers, a lever, a selectable switch, a ratchet mechanism or a rotationally or linearly movable mechanism. The first actuator is often adapted to provide audible, visual or tactile feedback during actuation so as to indicate the number of prostheses in the selected number. Often, each actuation of the first actuator retracts the outer sheath a distance equal to the length of one of the prostheses.

The second actuator may comprise an axially movable slider, a lever or a rotationally or linearly movable mechanism. The second actuator usually is movable through a predetermined distance and moving the second actuator through the predetermined distance creates the spacing with a predetermined length. The second actuator is not usually movable beyond the predetermined distance.

The second control may provide feedback to the operator when moved through the predetermined distance. The feedback may include a visual indicator associated with the second actuator or a detent engaged by the second actuator. Sometimes, after moving through the fixed distance, the second actuator is further movable to adjust the length of the spacing.

The apparatus may also comprise a pusher element disposed on the elongated flexible member that is adapted to prevent the prostheses from axially moving toward the proximal end of the elongated flexible member. An engaging member may also be included in the apparatus that is disposed near a distal end of the outer sheath. The engaging member can engage one or more of the prostheses so as to facilitate movement of the prostheses axially on the elongated flexible member. Sometimes the apparatus includes an expandable member coupled to the flexible member, and the prostheses are positionable on the expandable member for expansion therewith. The expandable member may be a balloon, and the sheath is often adapted to constrain the expandable member from expansion when positioned over the expandable member. Upon retraction of the sheath a selected portion of the expandable member is exposed for expansion of the selected number of prostheses. The apparatus also may comprise an indicator adapted to display the selected number of prostheses.

In another aspect of the present invention, an apparatus for delivering a prosthesis in a body lumen comprises an elongated flexible member having a proximal end, a distal end and at least a first and a second radially expandable prosthesis arranged axially in an unexpanded condition along the elongated flexible member, near the distal end. An outer sheath is slidably disposed over at least a portion of the prostheses and a control mechanism is coupled to the outer sheath and the elongated flexible member. The control mechanism has at least two settings, wherein upon actuation of the control mechanism in the first setting, the outer sheath is retracted to expose only the first prosthesis in the unexpanded condition and in the second setting the outer sheath is retracted to expose to expose both the first and the second prostheses in the unexpanded condition. Usually, upon actuation of the control mechanism in the first setting the outer sheath is retracted to expose only the first prosthesis and a preselected spacing between the first prosthesis and a distal end of the outer sheath is created. Actuation of the control mechanism in the second setting usually retracts the outer sheath to expose both the first and second prostheses and creates the preselected spacing between the second prosthesis and a distal end of the outer sheath.

The apparatus may further comprise a handle adjacent to the proximal end of the elongated flexible member. The handle may be held by an operator using one hand while simultaneously actuating the control mechanism with the fingers or thumb of the one hand. The control mechanism may also comprise a sliding switch selectable between the two settings, an axially movable actuator or two axially movable sliders. The apparatus may include an expandable member near the distal end of the elongated flexible member, which can be a balloon. The apparatus may have a pusher element disposed on the elongated flexible member that prevents the at least first and second prostheses from axially moving toward the proximal end of the elongated flexible member. The apparatus may further comprise an engaging member disposed near a distal end of the outer sheath that is adapted to engage one or more of the prostheses so as to facilitate movement of the prostheses axially on the elongated flexible member. The engaging member may be disposed a distance from a distal end of the sheath selected so that the engaging member engages only the second prosthesis when the sheath is positioned over both of the prostheses. The distance may be greater or shorter than the length of the first prosthesis.

The control mechanism is often operatively coupled with the outer sheath as well as the pusher element. The pusher element may be operatively coupled with the outer sheath. The control mechanism usually comprises an actuator for selectively coupling the pusher element to the outer sheath, and the actuator has a first position in which the pusher element is retractable with the sheath, and a second position in which the pusher element remains stationary as the sheath is retracted. The pusher element may comprise a pin slidably disposed in a slot in the outer sheath.

In another aspect of the present invention, a method for delivering prostheses to a body lumen comprises positioning an elongated flexible member at a first treatment site having a first lesion length, the elongated flexible member has a plurality of prostheses axially arranged thereon, covered by an outer sheath. A length setting is selected from a plurality of length settings on a control mechanism disposed on the elongated flexible member, and the length setting corresponds to a first group of prostheses for deployment. Actuating the control mechanism retracts the outer sheath a predetermined distance based on the length setting so that the first group of prostheses are unconstrained from expansion. The first group of prostheses are separated from any remaining prostheses and then deployed at the site of the lesion.

Selecting a length may comprise linearly or rotationally moving an actuator, actuating a pair of triggers pivotably coupled to a handle or actuating a lever or a switch. Actuating the control mechanism may comprise actuating it with a thumb or finger. Usually, deploying the first group comprises radially expanding an expandable member such as a balloon, disposed on the elongate flexible member. Separating the first group may comprise actuating the control mechanism so as to create a space free of the prostheses between a proximal prosthesis in the first group and a distal end of the outer sheath with the prostheses in the unexpanded condition. Actuating the control mechanism creates the space with a predetermined length and separating the first group further comprises adjusting the length of the space by actuating the control mechanism after creating the space with the predetermined length. Actuating the control mechanism comprises moving the actuator through a fixed distance thereby retracting the outer sheath a distance equal to the length of one prosthesis or a predetermined multiple thereof.

Selecting a length setting may comprise adjusting the position of a selector switch having a plurality of positions corresponding to different numbers of the prostheses to be included in the first group. Selecting a length may also include adjusting at least one of a plurality of axially movable sliders, the sliders being movable through a fixed distance. A first of the sliders often is movable through a first fixed distance and a second of the sliders is movable through a second fixed distance different than the first distance. Moving the first slider through the first fixed distance usually exposes a number of prostheses outside the sheath different than when moving the slider through the second fixed distance. Selecting a length setting may further comprise providing feedback indicating the selected length to an operator and the feedback may be a visual indicator or a detent engaged by the control mechanism. Actuating the control mechanism further comprises preventing the prostheses from axially moving toward the proximal end of the elongated flexible member.

In another aspect of the present invention, a method for delivering prostheses to a body lumen comprises positioning an elongated flexible member at a first treatment site having a first lesion length and the elongated flexible member has at least a first and a second prosthesis axially arranged thereon and they are covered by an outer sheath. A length setting is selected from at least two settings on a control mechanism disposed on the elongated flexible member, with the length setting corresponding to a number of prostheses for deployment. The control mechanism may be actuated in the first setting to retract the outer sheath to expose only the first prosthesis and create a pre-selected spacing between the first prosthesis and the second prosthesis. Actuating the control mechanism in the second setting retracts the outer sheath to expose both the first and the second prostheses and creates a pre-selected spacing between the second prosthesis and a distal end of the outer sheath. The prosthesis may then be deployed at the site of a lesion.

Actuating the control mechanism may comprise actuating it with a thumb or finger and selecting a length setting usually comprises adjusting the position of a selector switch having a plurality of positions corresponding to different numbers of the prostheses to be included in the first group. Selecting a length may comprise adjusting at least one of a plurality of axially movable sliders that are movable through a fixed distance. Often, selecting a length includes providing feedback that indicates the selected length to an operator. The feedback may include a visual indicator or a detent engaged by the control mechanism. Sometimes, actuating the control mechanism further comprises preventing the prostheses from axially moving toward the proximal end of the elongated flexible member. The control mechanism may be actuated by linearly moving it.

Selecting a length may comprise linearly moving an actuator, actuating a switch or moving an actuator with a thumb or finger. The control mechanism may also be actuated with a finger or thumb. Deploying the number of prostheses usually comprises radially expanding an expandable member such as a balloon that is disposed on the elongate flexible member.

Usually the prostheses are self-expanding or balloon expandable and carry a therapeutic agent adapted to be released therefrom, and often the therapeutic agent comprises an anti-restenosis agent. The prostheses usually have a length in the range from about 2 mm to about 20 mm, and often the prostheses comprise segments having a length about 3 mm to 10 mm. Sometimes the prostheses may have the same length, or they may have two or more lengths. Sometimes they may also have ends in engagement with one another prior to deployment.

These and other embodiments are described in further details in the following description related to the appended drawing figures.

### BRIEF DESCRIPTION OF THE DRAWINGS

FIG. 1 shows a perspective view of a stent delivery catheter having a control mechanism on the catheter handle adapted to retract the catheter sheath in accordance with one embodiment of the present invention.

FIGS. 2A-2E show selection and deployment of prosthetic stent segments to treat a lesion in accordance with an exemplary embodiment.

FIGS. 3A-3B show components of the handle illustrated in the stent delivery catheter of FIG. 1.

FIGS. 4A-4E show operation of the handle depicted in FIGS. 3A-3B.

FIG. 5 shows a perspective view of a stent delivery catheter with a control mechanism on the handle adapted to retract the catheter sheath, in accordance with another embodiment of the present invention.

FIGS. 6A-6C show the control mechanism depicted in FIG. 5 that is disposed on a handle and used to control stent selection and delivery.

FIGS. 7A-7F illustrate stent selection and deployment in an exemplary embodiment.

FIG. 8 illustrates another handle and control mechanism embodiment.

FIGS. 9A-9B show yet another handle and control mechanism embodiment.

FIGS. 10A-10B show still another handle and control mechanism embodiment.

FIGS. 11A-11B show another handle and control mechanism embodiment.

FIGS. 12A-12I show another handle and control mechanism embodiment.

FIG. 13A is a perspective view of yet another handle and control mechanism embodiment.

FIG. 13B is a bottom view of the handle illustrated in FIG. 13A.

### DETAILED DESCRIPTION OF THE INVENTION

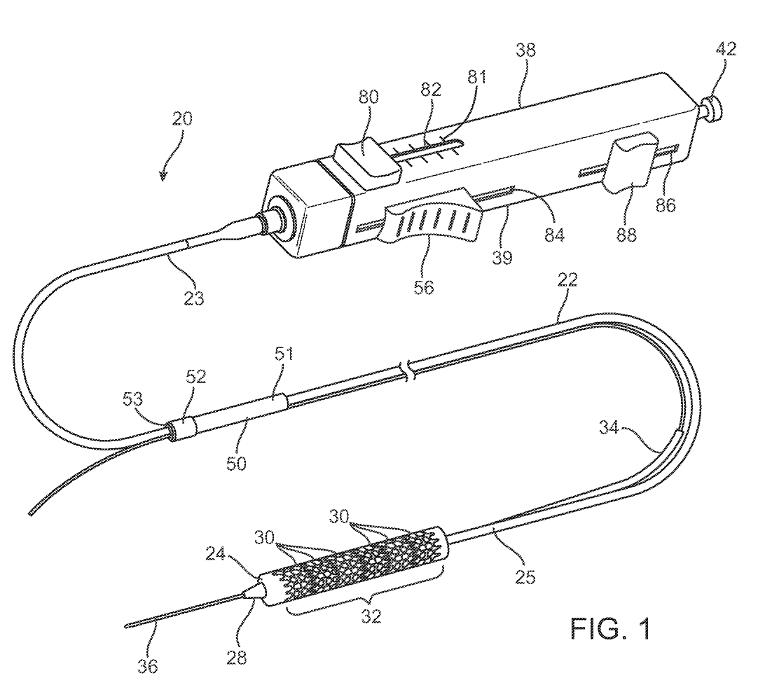

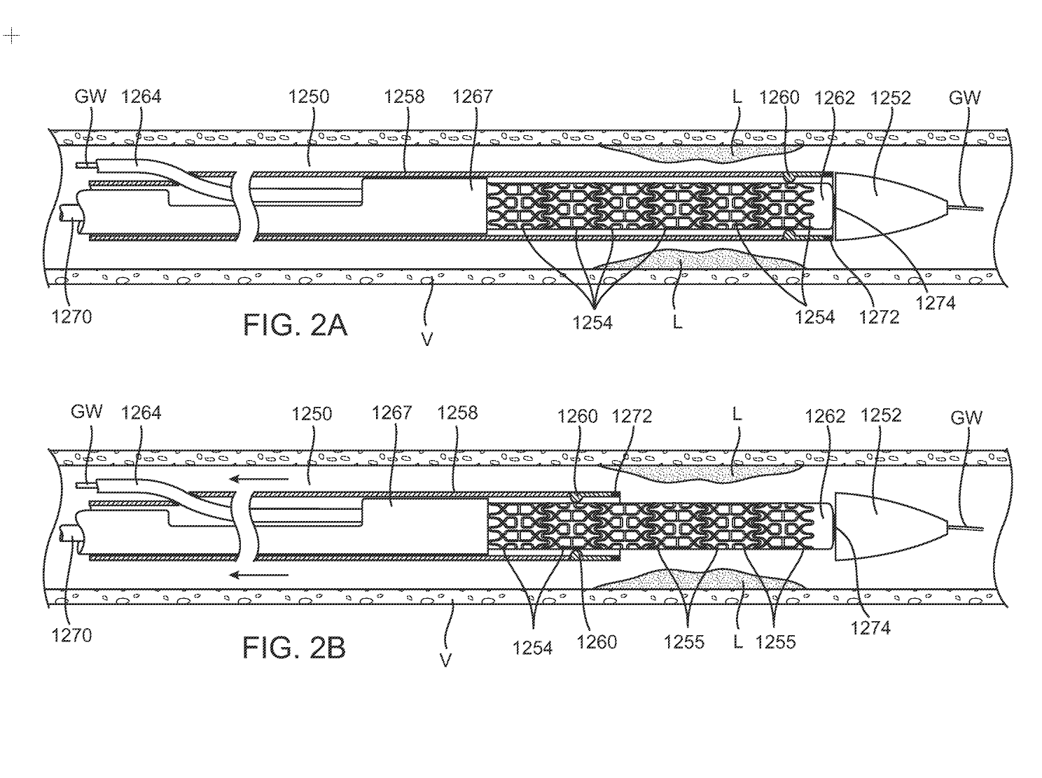

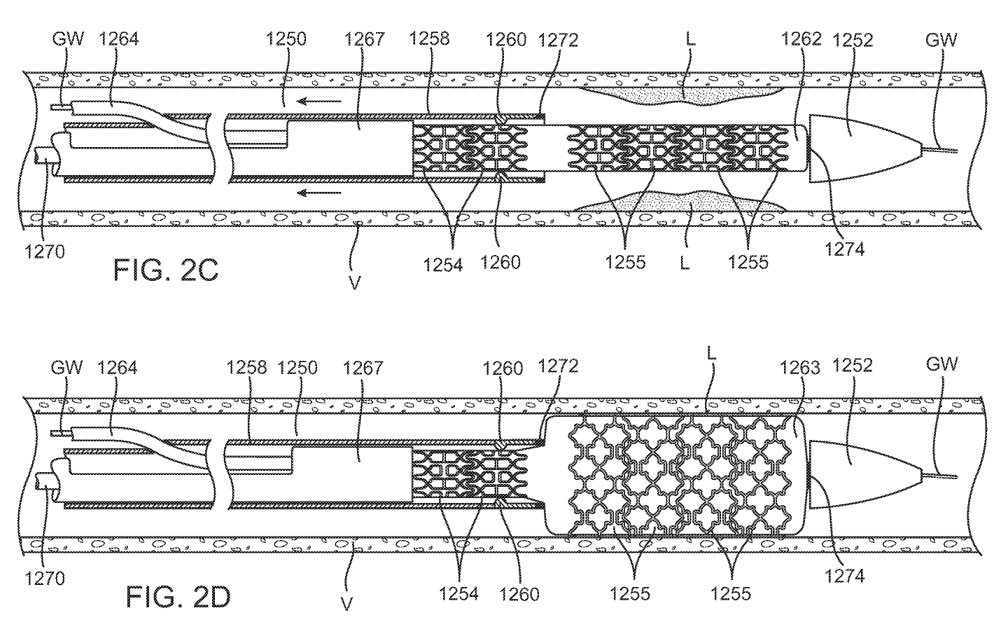

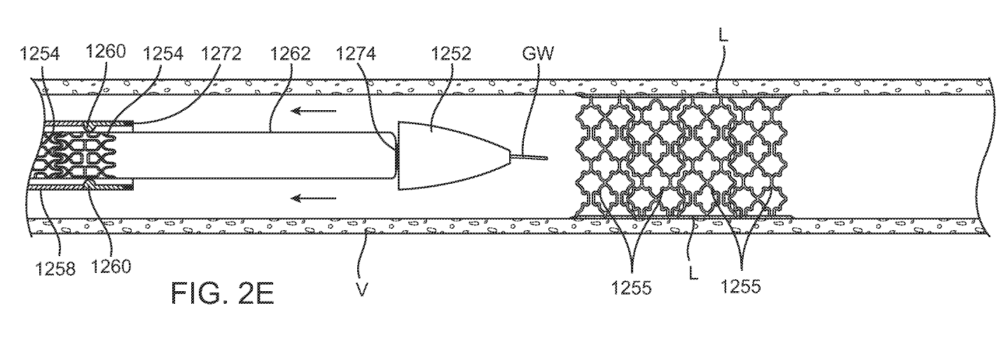

Referring now to FIG. 1, a stent delivery catheter 20 comprises a catheter shaft 22 with an outer sheath 25 slidably disposed over an inner shaft 1270 (in FIG. 2A). An inflatable balloon 24, is mounted on the inner shaft 1270 and is exposed by retracting sheath 25 relative to the inner shaft 1270. A tapered nosecone 28, composed of a soft elastomeric material to minimize trauma to the vessel during advancement of the delivery system 20, is attached distally of the inflatable balloon 24. Prosthesis 32 comprises a plurality of prosthetic segments 30 mounted over the inflatable balloon 24 for expansion. A guidewire tube 34 is slidably positioned through sheath 25 proximal to the inflatable balloon 24. A guidewire 36 is positioned slidably through guidewire tube 34, inflatable balloon 24 and nosecone 28, and extends distally thereof. FIG. 1 illustrates the stent delivery catheter 20 and FIG. 2A shows various elements of the delivery catheter 20 in greater detail.

FIG. 2A shows stent delivery catheter 1250 slidably advanced over the guidewire GW into the vessel V so that the nosecone 1252 is distal to the lesion L. Stent segments 1254 having interleaved ends in engagement with one another are disposed over expandable member 1262 and covered by outer sheath 1258. Expandable member 1262 is disposed over inner shaft 1270 having an inner lumen. In this embodiment, six stent segments 1254 are disposed on the stent delivery catheter 1250, each having a length approximately 6 mm long. Thus, in this embodiment, the delivery catheter 1250 is adapted to deliver a prosthesis having a length ranging from about 6 mm, up to 36 mm, in 6 mm increments. Other lengths and quantities of stent segments may be employed and this exemplary embodiment is not meant to limit the scope of the present invention. Pusher tube 1267 is engaged with the end of the proximal-most stent segment and prevents the stent segments 1254 from being axially displaced in the proximal direction as the outer sheath 1258 is retracted. Stent valve or engaging member 1260 is disposed on the inner surface of outer sheath 1258 and is adapted to engage one or more of the prosthetic segments 1254 so as to move the prosthetic segments 1254 axially along the inner shaft 1270 when the outer sheath 1258 is moved. Further details about the stent valve 1260 are described hereinbelow.

Referring to FIG. 1, a handle 38 is attached to a proximal end 23 of the sheath 25. The handle performs several functions, including retracting the sheath 25 thereby exposing prosthetic segments 30 and allowing the prosthetic segments 30 to be delivered. Additionally, the handle 38 creates a spacing between prosthetic segments 30 selected for delivery and the segments that will remain with the delivery catheter 20. This gap or spacing between segments permits proper balloon inflation and will be described hereinafter in further detail along with the handle structure and operation.

Handle 38 includes a housing 39 which encloses the internal components of the handle 38. Handle 38 allows a physician operator to select a retraction distance for outer sheath 25 which determines the length of the prosthesis (number of segments) to be deployed. The handle also permits connection of balloon 24 to an inflation source. The inner shaft 1270 is preferably fixed to the handle housing 39, while the outer sheath 25 is coupled to slide mechanism 56 so as to be retracted and advanced relative to handle 38. An adaptor 42 is attached to handle 38 at its proximal end and is fluidly coupled to the inner shaft 1270 in the interior of the housing of handle 38. The adaptor 42, preferably a Luer connector, is configured to be fluidly coupled with an inflation device which may be any commercially available balloon inflation device such as those sold under the trade name “Indeflator™,” manufactured by Abbott (formerly Guidant Corporation of Santa Clara, Calif.). The adaptor is in fluid communication with the inflatable balloon 24 via an inflation lumen (not shown) in the inner shaft 1270 to permit inflation of the inflatable balloon 24.

Additionally, a control mechanism on the handle 38 includes a slide mechanism 56 that translates along slot 84. Slide mechanism 56 is coupled with outer sheath 25 and is adapted to retract the sheath 25 a selected distance when slide 56 is retracted along slot 84. The distance is selected by sliding selector 80 along slot 82 to permit exposure of a pre-selected number of prosthetic segments on the distal end of delivery catheter 20. The selector mechanism 80 includes visual markers 81 so that an operator can easily determine how many stent segments have been selected. Additionally, selector mechanism 80 may provide audible or tactile feedback to the operator to facilitate operation of the stent delivery catheter 20 without requiring direction visualization during operation. The handle may also provide a visual indicator as well. The control mechanism also includes an additional spacer button 88 which slides along slot 86 to create a spacing between a prosthetic segment in the group of segments exposed from outer sheath 25 and a distal prosthetic segment remaining with outer sheath 25, after outer sheath 25 has been retracted. Further details on the operation of the slide mechanism 56 and selector 80 are described hereinbelow and additional details on materials and construction of other handles and control mechanisms are described in co-pending U.S. patent application Ser. No. 11/148,713, filed Jun. 8, 2005, (Attorney Docket No. 14592.4002), entitled “Devices and Methods for Operating and Controlling Interventional Apparatus,” U.S. patent application Ser. No. 10/746,466, filed Dec. 23, 2003 (Attorney Docket No. 0021629-002200US), entitled “Devices and Methods for Controlling and Indicating the Length of an Interventional Element,” and co-pending United States Publication No. 2005/0149159, entitled “Devices and Methods for Controlling and Indicating the Length of an Interventional Element,” which have previously been incorporated herein by reference.

Outer sheath 25 and guidewire 36 each extend through a slider assembly 50 slidably disposed on the catheter body 22 at a point between handle 38 and expandable member 24. The slider assembly 50 is adapted for insertion into and sealing with a hemostasis valve, such as on an introducer sheath or guiding catheter, while still allowing relative movement of the outer sheath 25 relative to the slider assembly 50. The slider assembly 50 includes a slider tube 51, a slider body 52, and a slider cap 53.

Outer sheath 25 may be composed of any of a variety of biocompatible materials, such as but not limited to polymers such as PTFE, FEP, polyimide, polyamines such as Nylon, or other thermoplastic elastomers including polyether bloc amides such as Pebax. The outer sheath 25 may be reinforced with a metallic or polymeric braid to resist radial expansion of inflatable balloon 24, and/or the like. Inflatable balloon 24 may be formed of a semi-compliant polymer such as polyether bloc amide (e.g. Pebax), polyamine (e.g. Nylon), polyurethane, polypropylene, PTFE or other suitable polymers. Additional aspects of the luminal prosthesis delivery system are described in U.S. patent application Ser. No. 10/306,813, filed Nov. 27, 2002 (Attorney Docket No. 021629-000320US); U.S. patent application Ser. No. 10/637,713 (Attorney Docket No. 021629-000340US), filed Aug. 8, 2003; U.S. patent application Ser. No. 10/738,666, filed Dec. 16, 2003 (Attorney Docket No. 021629-000510US); U.S. patent application Ser. No. 11/104,305, filed Apr. 11, 2005 (Attorney Docket No. 021629-003300US); and U.S. application Ser. No. 11/148,585, filed Jun. 8, 2005, the full disclosures of which are hereby incorporated by reference.

Delivery catheter 20 also includes an engaging member, also referred to as a stent valve 1260 disposed near the distal end of the outer sheath 1258, and an exemplary embodiment of this may be seen in FIG. 2A. In FIG. 2A, outer sheath 1258 rests in a fully distal position such that the distal end of the outer sheath 1258 is engaged with the proximal end of nosecone 1252 and the outer sheath 1258 covers all of the prosthetic segments 1254 which are disposed over expandable member 1262. Expandable member 1262 acts as a carrier which supports the prosthetic segments 1254.

Stent valve 1260, also referred to as engaging member 1260 contacts and engages prosthetic segments 1254. As shown in FIG. 2A, engaging member 1260 includes an annular flange configured to frictionally engage stent segments 1254 when sheath 1258 has been retracted and prosthetic segments 1254 are exposed. Thus, when outer sheath 1258 is retracted proximally, the engaging member 1260 engages the prosthetic segments 1254 and thereby moves them axially with outer sheath 1258 if pusher tube 1267 is allowed to move axially. The engaging member 1260 may be a polymeric or metallic material integrally formed with outer sheath 1258, or it may be a separate material bonded or otherwise mounted to the interior of the outer sheath 1258. The geometry of engaging member 1260 can also be toroidal with a circular or ovoid cross-section (like an O-ring) or the engaging member 1260 may have another cross-sectional shape such as triangular, trapezoidal, pyramidal, or other shapes as described in embodiments discussed more fully below. The engaging member 1260 can be a polymer such as silicone or urethane, sufficiently soft, compliant and resilient to provide frictional engagement with stent segments 1254, in some embodiments without damaging any coating deposited thereon, including therapeutic drug coatings and polymers or other carriers. The engaging member 1260 extends radially inwardly a sufficient distance to engage the exterior of stent segments 1254 with sufficient force to allow the stent segments which have not been selected for delivery to be retracted proximally with outer sheath 1258 (with pusher tube 1267 being allowed to move) so as to create a spacing relative to those stent segments selected for delivery. Other exemplary embodiments of stent valves or engaging members along with additional aspects of engaging member 1260 are described in U.S. patent application Ser. No. 10/412,714, filed Apr. 10, 2003 (Attorney Docket No. 021629-000330US); U.S. patent application Ser. No. 11/344,464, filed Jan. 30, 2006 (Attorney Docket No. 021629-003500US); and U.S. patent application Ser. No. 11/469,773 filed Sep. 1, 2006 (Attorney Docket No. 021629-004000US), the entire contents of which are incorporated herein by reference.

Prosthesis 32 is composed of one or more prosthetic segments 30. Prosthetic segments 30 are disposed over an inflation balloon 24. Each stent segment is about 2-20 mm in length, more typically about 2-12 mm in length and preferably being about 3-8 mm in length. Usually 2-20, more typically 2-10 and preferably 2-6 stent segments 30 may be positioned axially over the inflation balloon 24 and the inflation balloon 24 has a length suitable to accommodate the number of stent segments. Stent segments 30 may be positioned with ends in engagement with one another or a space may exist in between segments. Furthermore, the stent segments 30 may be deployed individually or in groups of two or more at a single treatment site within the vessel lumen. In preferred embodiments the adjacent ends have axially extending members that interleave with one another as described in copending U.S. patent application Ser. No. 10/738,666 (Attorney Docket No. 021629-000510US) filed Dec. 16, 2003.

Prosthetic stent segments 30 are preferably composed of a malleable metal such as stainless steel or cobalt-chromium alloy, so they may be plastically deformed by inflation balloon 24 as they are radially expanded to a desired diameter in the vessel at the target treatment site. The stent segments 30 may also be composed of an elastic or superelastic shape memory alloy such as Nitinol so that the stent segments 30 self-expand upon release into a vessel by retraction of the outer sheath 25. In this case, an inflation balloon 24 is not required but may still be used for pre-dilation of a lesion or augmenting expansion of the self-expanding stent segments (e.g. post-dilation or tacking). Other materials such as biocompatible polymers may be used to fabricate prosthetic stent segments and these materials may further include bioabsorbable or bioerodable properties.

Stent segments 30 may have any of a variety of common constructions, such as but not limited to those described in U.S. patent application Ser. No. 10/738,666 filed Dec. 16, 2003 (Attorney Docket No. 02169-000510US), which was previously incorporated by reference. Constructions may include for example, closed cell constructions including expansible ovals, ellipses, box structures, expandable diamond structures, etc. In addition, the closed cells may have complex slotted geometries such as H-shaped slots, I-shaped slots, J-shaped slots, etc. Suitable open cell structures include zigzag structures, serpentine structures, and the like. Such conventional stent structures are well described in the patent and medical literature. Specific examples of suitable stent structures are described in the following U.S. Patents, the full disclosures of which are incorporated herein by reference: U.S. Pat. Nos.: 6,315,794; 5,980,552; 5,836,964; 5,527,354; 5,421,955; 4,886,062; and 4,776,337.

In preferred embodiments, prosthetic stent segments 30 may be coated, impregnated, infused or otherwise coupled with one or more drugs that inhibit restenosis, such as Rapamycin, Everolimus, Biolimus A9, Paclitaxel, analogs, prodrugs, or derivatives of the aforementioned, or other suitable agents, preferably carried in a durable or bioerodable carrier of polymeric or other suitable material. Alternatively, stent segments 30 may be coated with other types of drugs or therapeutic materials such as antibiotics, thrombolytics, anti-thrombotics, anti-inflammatories, cytotoxic agents, anti-proliferative agents, vasodilators, gene therapy agents, radioactive agents, immunosuppressants, chemotherapeutics and/or stem cells. Such materials may be coated over all or a portion of the surface of stent segments 30, or stent segments 30 may have a porous structure or include apertures, holes, channels, or other features in which such materials may be deposited.

Some embodiments may include combinations of therapeutic agents to achieve synergistic results, such as using a drug that inhibits stenosis like Biolimus A9 and an anti-thrombotic agent such as Heparin so as to potentially reduce the latent thrombosis rate that may be associated with some drug eluting stents. Examples of anticoagulants which are commonly used to control thrombosis include Heparin, vitamin K antagonists, Warfarin (Coumadin), acenocoumarol, phenprocoumon and phenindione, as well as others reported in the medical and patent literature. Additionally, such anti-thrombotic agents could be delivered to the treatment region using time release methods (such as a polymer matrix) to control delivery of the agent over time.

Referring now to FIGS. 2A-2E, the deployment of selected prosthetic segments to treat a lesion is shown in accordance with an exemplary embodiment. While the embodiment will be described in the context of a coronary artery stent procedure, it should be understood that the invention may be employed in any variety of blood vessels and other body lumens in which stents or tubular prostheses are deployed, including the carotid, femoral, iliac and other arteries and veins, as well as non-vascular body lumens, such as the ureter, urethra, fallopian tubes, the hepatic and biliary ducts and the like. A guide catheter (not illustrated) is first inserted into a peripheral artery such as the femoral artery, typically using a percutaneous procedure such as the Seldinger technique or by surgical cutdown, and then advanced to the ostium of the right or left coronary artery. Guidewire GW is then inserted through the guiding catheter and advanced into the target vessel V where a lesion L to be treated is located. The proximal end of guidewire GW is then inserted through nosecone 1252 and guidewire tube 1264 which is outside the patient's body.

FIG. 2A shows stent delivery catheter 1250 slidably advanced over the guidewire GW into the vessel V so that the nosecone 1252 is distal to the lesion L. Stent segments 1254 having interleaved ends in engagement with each other are disposed over expandable member 1262 and covered by outer sheath 1258. In this embodiment, six stent segments 1254 are disposed on the stent delivery catheter 1250, each having a length approximately 6 mm long. Thus, in this embodiment, the delivery catheter 1250 is adapted to deliver a prosthesis having a length ranging from about 6 mm long up to about 36 mm long, in 6 mm increments. Other lengths and quantities of stent segments may be employed and this exemplary embodiment is not meant to limit the scope of the present invention. Pusher tube 1267 is engaged with the end of the proximal-most stent segment and prevents the stent segments 1254 from being axially displaced in the proximal direction as the outer sheath 1258 is retracted. Stent valve 1260 is disposed on the inner diameter of outer sheath 1258 and facilitates deployment of stent segments 1254 by engaging stent segments 1254 to allow creation of spacing between the stent segments to be deployed and those to be retained on the catheter during each deployment. Pusher tube 1267 prevents stent segments 1254 from being retracted proximally as outer sheath 1258 is retracted proximally.

The length of the lesion to be treated is typically visualized by introducing contrast media into the target vessel V and observing the resulting image under a fluoroscope. Radiopaque markers 1272, 1274, one at the distal end of the balloon 1262 and one at the distal end of the outer sheath 1258 may be used to visualize the length of stent segments exposed for deployment relative to the target lesion. A number of stent segments 1255 are selected to traverse the length of the lesion L as depicted in FIG. 2B. Outer sheath 1258 is axially retracted in the proximal direction by means of a control mechanism (e.g. 56 in FIG. 1) on the proximal end of the delivery catheter 1250. As outer sheath 1258 is pulled back, the selected number of stent segments 1255 are exposed. Pusher tube 1267 is operably coupled with outer sheath 1258 such that during retraction of the outer sheath 1250 to expose stent segments 1255, pusher tube 1267 remains stationary and acts as a backstop to prevent proximal motion of the stent segments 1254, 1255 as the stent valve 1260 passes over them. In FIG. 2B, four segments 1255 having a total length of 24 mm have been selected to traverse an appropriately sized lesion. Additional stent segments 1254 remain covered by outer sheath 1258 unless they are selected for delivery if the initial quantity exposed is insufficient to cover the target lesion.

Referring now to FIG. 2C, a spacing is created between the stent segments selected for delivery 1255 and the segments 1254 remaining with the delivery catheter 1250. In FIG. 2C, outer sheath 1258 is again retracted by a control mechanism (e.g. 56 in FIG. 1) on the proximal end of the catheter. Slider 56 is operably coupled with outer sheath 1258, therefore as slider 56 is retracted proximally, outer sheath 1258 is also retraced by the slider 56. Pusher tube 1267 is operably coupled with the outer sheath 1258 so that as outer sheath 1258 is retracted by proximal retraction of slider 56, pusher tube 1267 retracts with the outer sheath 1258. Stent valve 1260 engages stent segments 1254 remaining with the delivery catheter and draws this group of stent segments proximally, away from the stent segments 1255 selected for delivery, thereby creating a spacing of about 2 mm to 6 mm. Additionally, a spacing is created between the distal end of outer sheath 1258 and the proximal-most end of the stents 1255 selected for delivery. This spacing typically has an axial length of about 1 mm to 3 mm and is necessary for a proximal taper, approximately 1 mm to 3 mm long, to form on the balloon during balloon inflation while minimizing the amount of the exposed balloon over which no stent segments are positioned for expansion. Additional details on the control mechanism are described below

In FIG. 2D, the selected stent segments 1255 are deployed. Expandable member 1262, typically a balloon, is inflated with a fluid such as contrast media and/or saline to achieve an expanded diameter 1263. Radial expansion of expandable member 1262 correspondingly expands stent segments 1255 against the vessel wall across lesion L. Outer sheath 1258 constrains the proximal portion of inflatable member 1262 and prevents deployment of the stent segments 1254 remaining with the delivery catheter 1250. After stent segments 1255 are deployed, expanded member 1263 is deflated and removed from the deployed stent segments 1255, leaving stent segments 1255 in a plastically deformed, expanded configuration in the vessel V, at the site of the lesion, L. This is illustrated in FIG. 2E. Stent segments 1254 remain with the delivery catheter 1250 and both are removed and retracted from the vessel V.

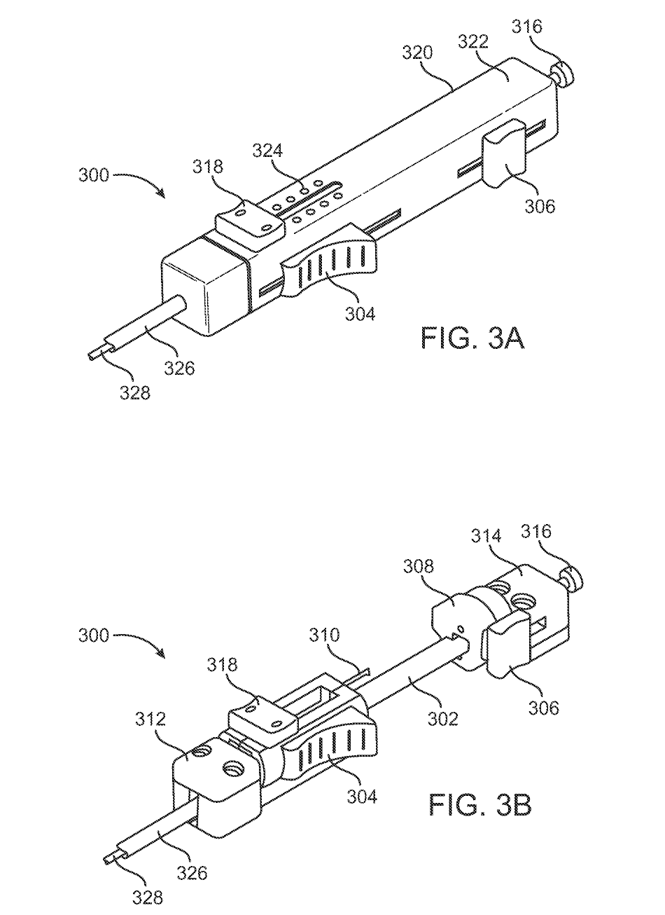

FIGS. 3A-3B show perspective views of a handle and control mechanism embodiment that may be utilized to control stent segment deployment according to an embodiment of the present invention. FIG. 3A highlights the proximal end of a stent delivery catheter 300, where the handle 320 is located. The stent delivery catheter 300 includes a handle 320 having a housing 322 enclosing components of the handle 320 and providing an ergonomic surface for a physician operator to grasp during use. An adapter 316 on the proximal end of handle 320 allows an inflation device to be fluidly connected with the catheter and an inflatable member on the delivery catheter 300 distal end (not shown).

An outer sheath 326 and pusher tube 328 are coupled with the distal end of handle 320. Operator controls 304, 306 and 318 extend from the catheter handle housing 322 and permit an operator to select a number of stent segments for deployment. Slider 318 is calibrated and allows a physician to set the number of stent segments for deployment. Detents and/or visual markers along the ratchet lever slot 324 provide tactile, audible and/or visual feedback to facilitate the operator with stent segment selection. This is particularly useful when the physician operator is simultaneously observing a fluoroscopic viewing screen while operating the handle during the stenting procedure.

Once the number of stent segments to be deployed has been determined by adjusting slider 318, retraction lever 304 is then retracted proximally. Retraction lever 304 is coupled with outer sheath 326, so outer sheath 326 is also displaced, exposing stent segments on the distal end of delivery catheter 300. Retraction lever 304 can only be retracted a fixed distance, which is determined by the position of slider 318. Once retraction of retraction lever 304 is complete and the desired number of stent segments are exposed from the sheath 326, pusher release lever 306 can be depressed to unlock it and then lever 306 is retracted creating a spacing between stent segments to be deployed and those remaining with the outer sheath 326. This spacing has previously been described and is required for proper balloon inflation during stent segment deployment.

After outer sheath 326 has been retracted to expose stent segments to be deployed and a proper spacing has been created, an expandable member is inflated and the stent segments are radially expanded and implanted at the target lesion site. The expandable member is then deflated and the stent delivery catheter is removed from the lesion site. This has been described previously, with respect to FIGS. 2A-2E.

FIG. 3B is a perspective view of the handle 320 shown in FIG. 3A with portions of the housing 322 removed to show internal components of the handle 320. In addition to the components previously described with respect to FIG. 3A, the handle mechanism also includes an actuation shaft 310 which is coupled with ratchet lever 318. As ratchet lever 318 is retracted, so too is actuation shaft 310, thus creating a mechanical stop, fixing the distance that activation lever 304 can be retracted. A rigid frame element 302 serves as a chassis for the handle 320 over which handle components can slide. Pusher assembly 308 also slides axially over rigid frame element 302 after actuation lever 304 is retracted and actuation shaft 310 has mated with pusher assembly 308. Pusher assembly 308 is retracted after pusher release lever 306 is depressed, creating the spacing necessary for proper balloon inflation. This is achieved because both outer sheath 326 (coupled with activation lever 304) and pusher tube 328 (coupled with pusher assembly 308) are retracted together as described more fully below.

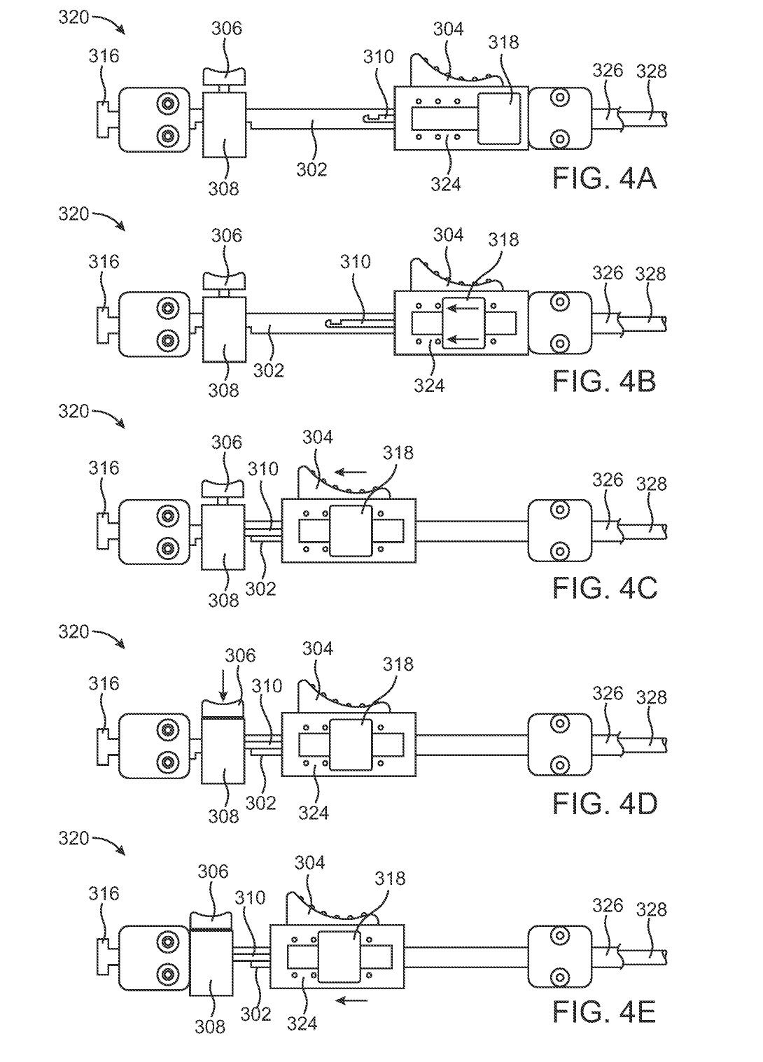

FIGS. 4A-4E illustrate operation of the control handle shown in FIGS. 3A-3B. Handle 320 is illustrated in FIG. 4A with housing 322 removed so that operation of the internal components may be seen. Handle 320 is depicted in FIG. 4A in an initial position, prior to operation. In FIG. 4A, handle 320 includes a rigid frame element 302 which serves as a chassis for handle components and also as an axis over which some of the handle components are slidably disposed including activation lever 304. Activation lever 304 is retracted after ratchet lever 318 has been adjusted along ratchet lever slot 324, setting a fixed displacement distance for activation lever 304 by exposing actuation shaft 310 a given distance. Actuation lever 304 is operably coupled with outer shaft 326 so that retracting actuation lever 304 a set distance will correspondingly displace outer sheath 326. Handle 320 also includes pusher assembly 308 and pusher release lever 306 which control motion of the pusher tube 328. Adaptor 316 is fluidly coupled with the proximal handle end.

In FIG. 4B, the first step of handle operation is illustrated. Ratchet lever 318 is retracted along the calibrated ratchet lever slot 324 a fixed distance. This distance is calibrated to permit a given number of stent segments to be exposed for delivery to a lesion of known length. As ratchet lever 318 is retracted, actuation shaft 310 is displaced along with the ratchet lever 318. Ratchet lever slot 324 contains detents or visual indicators in preferred embodiments that provide an operator with tactile, visual and/or audible feedback so that the operator can easily determine how far back the ratchet lever 318 has been retracted, which corresponds with how many stent segments are to be deployed.

Once ratchet lever 318 has been retracted, activation lever 304 is retracted, as shown in FIG. 4C. Activation lever 304 is retracted until actuation shaft 310 engages with pusher assembly 308. Actuation lever 304 is coupled with outer sheath 326 therefore, retraction of actuation lever 304 retracts outer sheath 326 a fixed distance, exposing a given number of stent segments on the distal end of the catheter.

After the selected number of stent segments are exposed from the outer sheath 326, a spacing must be created between the proximal-most end of the stent segments selected for delivery and the distal end of the outer sheath 326 and any remaining stent segments. This is illustrated in FIGS. 4D-4E. In FIG. 4D, pusher release lever 306 is depressed, releasing pusher assembly 308. In FIG. 4E, pusher assembly 308 is retracted proximally. Pusher assembly 308 is coupled with activation lever 304 by actuation shaft 310, therefore as pusher assembly 308 is retracted, so too is activation lever 304 and outer sheath 326. Simultaneously, pusher tube 328 is also coupled with pusher assembly 308, so pusher tube 328 retracts too. Retraction of both pusher tube 328 and outer sheath 328 allow a spacing to be created which is necessary for proper balloon inflation. Once the spacing is created, the stent segments selected for delivery may be deployed and the delivery catheter removed.

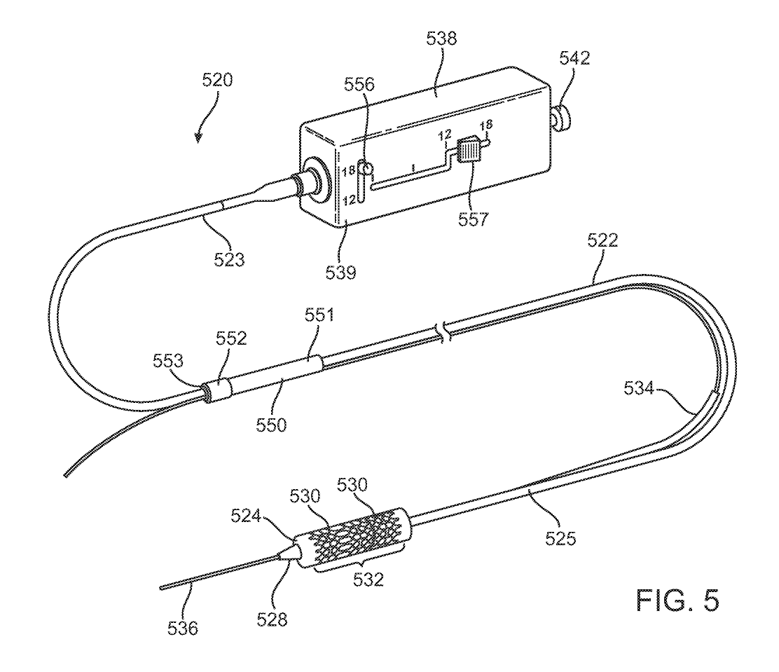

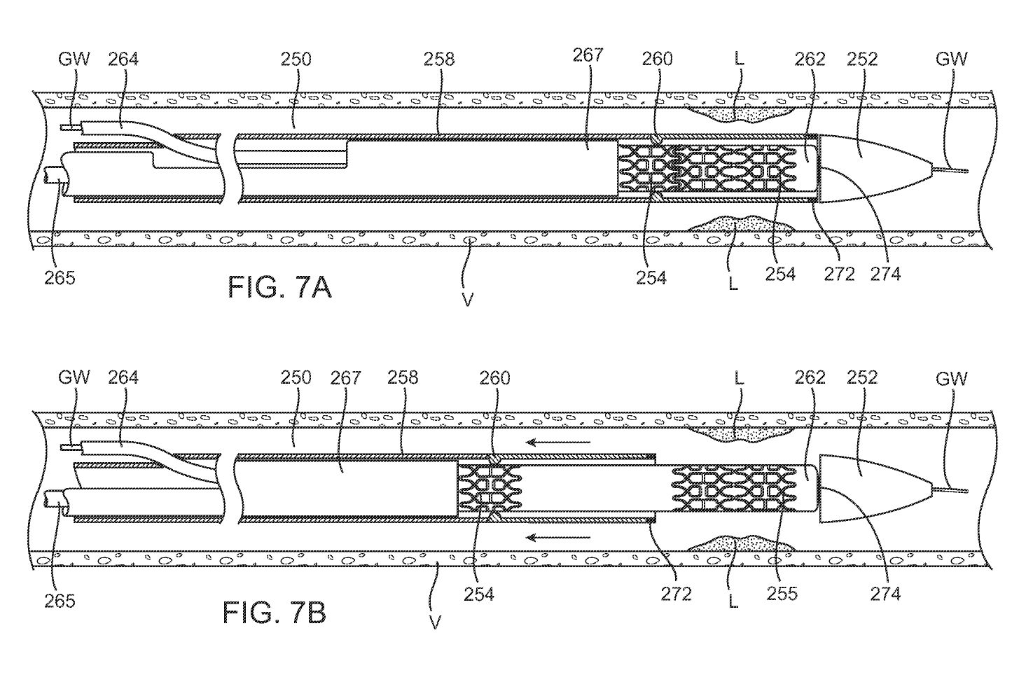

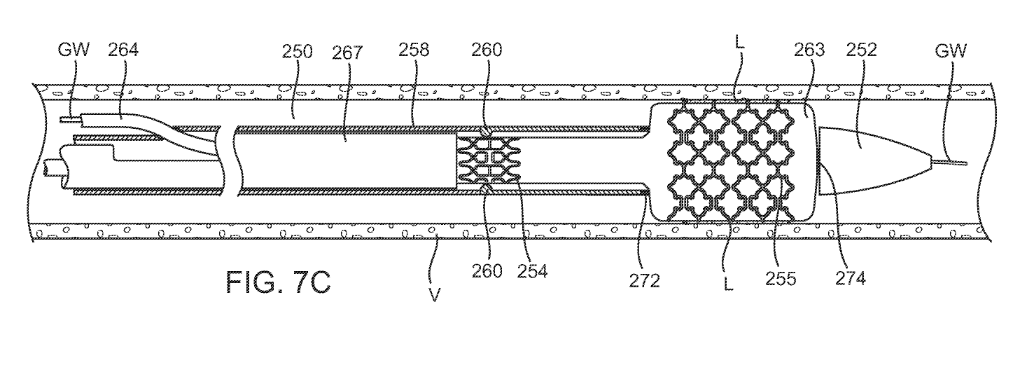

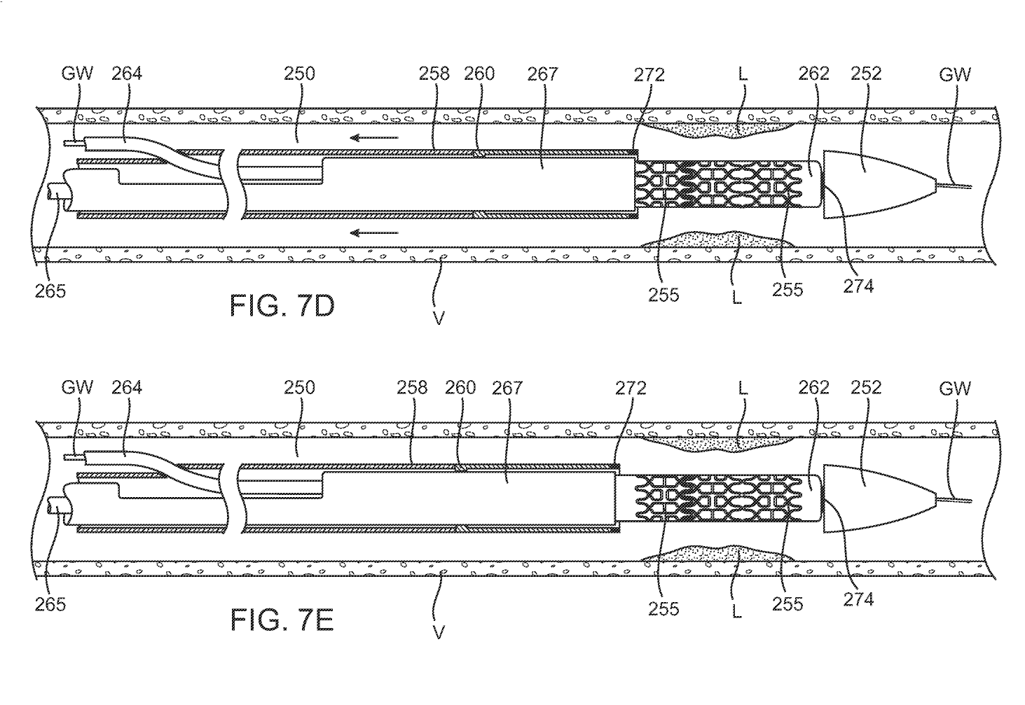

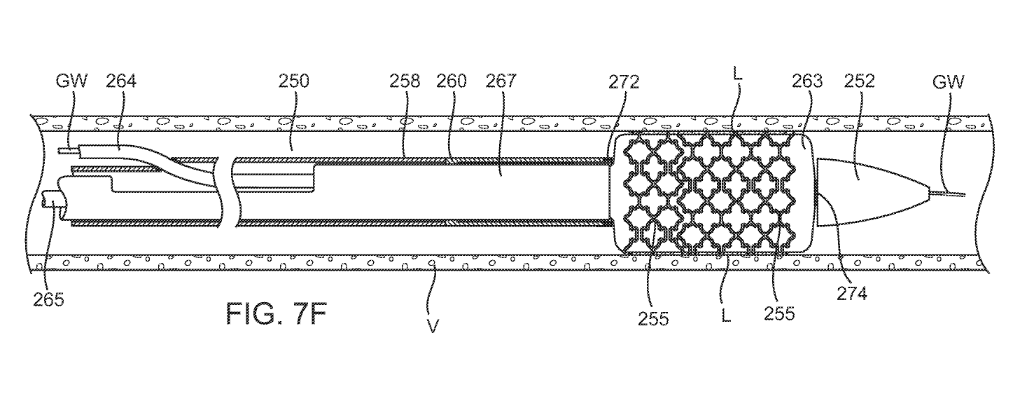

Referring now to FIG. 5, another embodiment of a stent delivery catheter is illustrated. The embodiment of FIG. 5 is different from the previous embodiment in several aspects. This embodiment only allows the user to select between two lengths of prostheses to deploy and does not require a separate step to create a spacing between stent segments selected for deployment and those remaining with the delivery catheter. In FIG. 5, a stent delivery catheter 520 comprises a catheter shaft 522 with an outer sheath 525 slidably disposed over an inner shaft 265 (FIG. 7A). An inflatable balloon 524, is mounted on the inner shaft 265 and is exposed by retracting sheath 525 relative to the inner shaft 265. A tapered nosecone 528, composed of a soft elastomeric material to minimize trauma to the vessel during advancement of the delivery system 520, is attached distally of the inflatable balloon 524. Prosthesis 532 comprises a plurality of prosthetic segments 530 mounted over the inflatable balloon 524 for expansion. A guidewire tube 534 is slidably positioned through sheath 525 proximal to the inflatable balloon 524. A guidewire 536 is positioned slidably through guidewire tube 534, inflatable balloon 524 and nosecone 528, and extends distally thereof.

A handle 538 is attached to a proximal end 523 of the sheath 525. The handle 538 performs several functions, including retracting the sheath 525 thereby exposing prosthetic segments 530 and allowing the prosthetic segments 530 to be delivered. Additionally, the handle 538 creates a gap between prosthetic segments 530 selected for delivery and the segments that will remain with the delivery catheter 520. This gap or spacing between segments permits proper balloon inflation and has previously been described.

Handle 538 includes a housing 539 which encloses the internal components of the handle 538. Handle 538 allows an operator to select a fixed distance for outer sheath 525 retraction followed by retraction of the outer sheath 525 by the selected fixed distance. The handle also permits connection of balloon 524 to an inflation source. The inner shaft 265 is preferably fixed to the handle body 539, while the outer sheath 525 is able to be retracted and advanced relative to handle 538. An adaptor 542 is attached to handle 538 at its proximal end and is fluidly coupled to the inner shaft 265 in the interior of the housing of handle 538. The adaptor 542, preferably a Luer connector, is configured to be fluidly coupled with an inflation device which may be any commercially available balloon inflation device such as those previously described above with respect to FIG. 1. The adaptor 542 is in fluid communication with the inflatable balloon 524 via an inflation lumen in the inner shaft 265 (FIG. 7A) to permit inflation of the inflatable balloon 524.

Additionally, a control mechanism 556 on handle 538 is coupled with outer sheath 525 and is adapted to select a fixed distance for retraction of outer sheath 525. The fixed distance is selected to expose a number of prosthetic segments and also allow creation of a spacing between a prosthetic segment in the exposed group of stent segments and any stent segments remaining with the outer sheath 525 or with the distal end of outer sheath 525. A slider 557 forms a portion of the control mechanism 556 and is also disposed on the handle 538 allowing a user to retract the outer sheath 525 a distance fixed by the position of the control mechanism 556. Further details on the operation of the control mechanism 556 and slider 557 are described below and additional details on handle materials and construction are described in co-pending United States Patent Applications and Publications which have previously been incorporated herein by reference.

The outer sheath 525 and guidewire 536 each extend through a slider assembly 550 located on the catheter body 522 at a point between its proximal and distal ends. The slider assembly 550 is adapted for insertion into and sealing with a hemostasis valve, such as on an introducer sheath or guiding catheter, while still allowing relative movement of the outer sheath 525 relative to the slider assembly 550. The slider assembly 550 includes a slider tube 551, a slider body 552, and a slider cap 553.

Outer sheath 525 generally takes the same form as outer sheath 25 in FIG. 1. Outer sheath 525 may be composed of any of a variety of biocompatible materials, such as but not limited to a polymer such as PTFE, FEP, polyimide, polyamides including Nylon or other thermoplastic elastomers such as polyether bloc amides including Pebax. The outer sheath 525 may be reinforced with a metallic or polymeric braid to resist radial expansion of inflatable balloon 524, and/or the like. Inflatable balloon 524 generally takes the same form as balloon 24 in FIG. 1 and may be formed of a semi-compliant polymer such as Pebax, Nylon, polyurethane, polypropylene, PTFE or other suitable polymers. Additional aspects of the luminal prosthesis delivery system are described in U.S. Patent Applications and Publications which have previously been incorporated by reference.

Prosthesis 532 is composed of one or more prosthetic segments 530. Prosthetic stent segments 530 are disposed over an inflation balloon 524. Each stent segment is about 2-20 mm in length, more typically about 2-15 mm in length and preferably being about 2-12 mm in length. Usually 2-20, more typically 2-10 and preferably 2-3 stent segments 30 may be positioned axially over the inflation balloon 524 and the inflation balloon 524 has a length suitable to accommodate the number of stent segments. Stent segments 530 may be positioned with interleaving ends in engagement with each other, or a space may exist in between segments. Furthermore, the stent segments 530 may be deployed individually or in groups of two or more at a single treatment site within the vessel lumen.

Stent segments 530 are preferably composed of a malleable metal so they may be plastically deformed by inflation balloon 524 as they are radially expanded to a desired diameter in the vessel at the target treatment site. The stent segments 530 may also be composed of an elastic or superelastic shape memory alloy such as Nitinol so that the stent segments 530 self-expand upon release into a vessel by retraction of the outer sheath 525. In this case, an inflation balloon 524 is not required but may still be used for pre-dilation of a lesion or augmenting expansion of the self-expanding stent segments. Other materials such as biocompatible polymers may be used to fabricate prosthetic stent segments and these materials may further include bioabsorbable or bioerodable properties.

Prosthetic stent segments 530 generally take the same form as stent segments 30 in FIG. 1. Stent segments 530 may have any of a variety of common constructions as discussed previously and in preferred embodiments stent segments 530 may be coated, impregnated, infused or otherwise coupled with one or more therapeutic agents. These aspects have been previously discussed above with respect to stent segments 30 in FIG. 1.

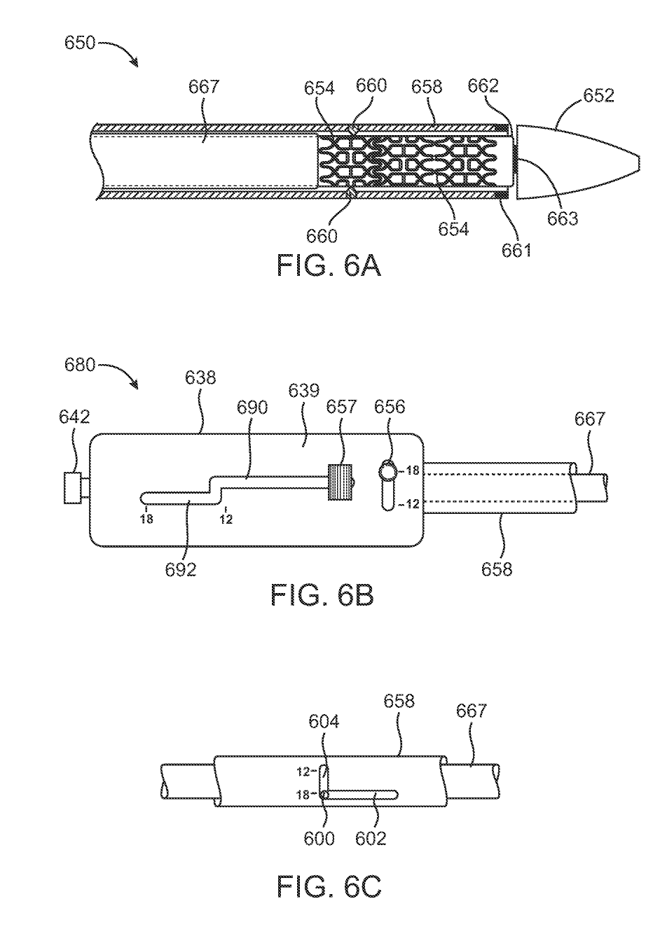

FIGS. 6A-6C illustrate the operation of the stent delivery system shown in FIG. 5. FIG. 6A highlights the distal end of catheter delivery system 650. In this embodiment two stent segments 654 are axially disposed over expandable member 662 while outer sheath 658 covers and protects the stent segments 654 prior to deployment. A pusher tube 667 acts as a backstop and prevents axial displacement of the stent segments 654 in the proximal direction when outer sheath 658 is moved over the stent segments 654. In this preferred embodiment, one stent segment 658 is 6 mm long while the second and distal-most stent segment 654 is 12 mm long, thus permitting delivery of a prosthesis having a total length either 12 mm long or 18 mm long. Stent valve 660 is disposed on the inner diameter of outer sheath 658 at an axial location selected so that is engages the proximal stent 654 when the outer sheath 658 is in its initial distal-most position.

FIG. 6B illustrates the proximal handle end 680 of the stent delivery system shown in FIG. 5. Referring to FIG. 6B, a control mechanism 656 is disposed on handle 638 and is attached with the outer sheath 658 proximal end. Catheter handle 638 is also operably coupled with the pusher tube 667. The handle 638 has a housing 639 which encloses the internal components of the handle 638. Handle 638 performs several functions including selecting a fixed distance for retraction of outer sheath 658 as well as retracting the outer sheath 658 the selected fixed distance. The handle 638 also permits connection of expandable member 662 to an inflation source via a connector 642. A catheter inner shaft 265 (FIG. 7A) is preferably fixed to the handle body 639, while the outer sheath 658 is able to be retracted and advanced relative to handle 638. An adaptor 642 is attached to handle 638 at its proximal end and is fluidly coupled to the inner shaft in the interior of the housing of handle 638. The adaptor 642, preferably a Luer connector, is configured to be fluidly coupled with inflation devices which have previously been discussed. The adaptor 642 is in fluid communication with the inflatable balloon 662 via an inflation lumen in the inner shaft 265 to permit inflation of the inflatable balloon 662.

Additionally, a control mechanism 656 on handle 638 is coupled with outer sheath 658 and is adapted to select a fixed distance for retraction of outer sheath 658. The fixed distance is selected to expose a number of prosthetic stent segments and also creates a spacing between a prosthetic segment in the group of prosthetic segments exposed and a distal prosthetic segment remaining with the outer sheath 658 or the distal end of outer sheath 658. The control mechanism 565 also comprises a slider 657 which travels along slots 690 and 692 and is disposed on handle 638 allowing a user to retract the outer sheath 658 a distance fixed by the position of the control mechanism 656. Typical handle materials include metals such as stainless steel as well as thermoplastics commonly used for such applications such as ABS, PVC, Ultem™ and polycarbonate.

FIG. 6C illustrates how the pusher tube 667 and outer sheath 658 are operably connected. A protruding locking element 600, such as a pin, extends from the pusher tube 667 through an L-shaped slot 602, 604 in outer sheath 658. When the locking element 600 is in the “18” position, as controlled by control mechanism 656, outer sheath 658 is free to translate axially along slot 602 and relative to pusher tube 667. The length of slot 602 is a fixed distance adequate to allow outer sheath 658 to be retracted until stent valve 660 has cleared the proximal segment 654. At that point the pusher tube 667 and outer sheath 658 retract together until both stent segments 654 are exposed. In this case, the outer sheath 658 would be able to translate at least 18 mm, enough to expose a 12 mm stent segment 654 as well as a 6 mm stent segment, plus an additional amount, approximately 1 mm to 3 mm to allow creation of a spacing between stent segments 654 and the distal end of outer sheath 658. When the locking element 600 is in the “12” position, as determined by control mechanism 656, outer sheath 658 and pusher tube 667 are locked together and therefore, as the outer sheath 658 is pulled back proximally to expose the 12 mm long stent segment 654, the pusher tube moves with the outer sheath 658 allowing proximal segment 654 to be retracted proximally with the outer sheath 658. The position of locking element 600 is adjusted by switching control mechanism 656 from either the “12” to “18” positions. Thus, switching control mechanism 656 pivots outer sheath 658 relative to pusher tube 667.

Slider mechanism 657 is used to axially displace outer sheath 658 once a fixed distance has been selected using control mechanism 656. For example, when control mechanism 656 is in the “12” position, slider 657 is drawn back by the operator's thumb along slot 690. Slot 690 has a length selected such that as slider 657 is drawn back along slot 690, outer sheath 658 is also displaced a distance adequate to expose the 12 mm stent segment 654 and to provide a spacing to be created between stent segments 654 automatically, without an additional step. Additionally, when control mechanism 656 is in the “18” position, slider 657 is pulled back until it reaches the end of slot 690 and then slight lateral motion translates the slider 657 into slot 692. The slider 657 may then be drawn back further, to permit exposure of both 12 mm and 6 mm long stent segments 654, as well as automatic creation of a small spacing in between the distal end of the outer sheath 658 and the proximal end of the 6 mm stent 654, so as to permit proper inflation of balloon 662 later on. The handle 638 is designed to permit single handed use while a physician operator is viewing a fluoroscope monitor.

FIGS. 7A-7F illustrate the deployment of stent segments in a vessel using the exemplary embodiment of FIG. 5 and FIGS. 6A-6C. In a stenting procedure, a guide catheter (not illustrated) is first inserted into a peripheral artery such as the femoral artery, typically using a percutaneous procedure such as the Seldinger technique or by surgical cutdown, and then advanced to the ostium of the right or left coronary artery. Guidewire GW is then inserted through the guiding catheter and advanced into the target vessel V where a lesion L to be treated is located. The proximal end of guidewire GW is then inserted through nosecone 252 and guidewire tube 264 which is outside the patient's body.

FIG. 7A shows stent delivery catheter 250 slidably advanced over the guidewire GW into the vessel V so that the nosecone 252 is distal to the lesion L. Stent segments 254 having interleaved ends in engagement with one another are disposed over expandable member 262 and covered by outer sheath 258. In this embodiment, two stent segments 254 are disposed on the stent delivery catheter 250, a proximal segment having a length 6 mm long and a distal segment 12 mm long. Thus, in this embodiment, the delivery catheter 250 is adapted to deliver a prosthesis having a total length of either 12 mm or 18 mm. Other lengths and quantities of stent segments may be employed and this exemplary embodiment is not meant to limit the scope of the present invention. Pusher tube 267 is engaged with the end of the proximal-most stent segment 254 and prevents the stent segments 254 from being axially displaced in the proximal direction as the outer sheath 258 is retracted. Stent valve 260 is disposed on the inner diameter of outer sheath 258 and facilitates separation of stent segments 254 during deployment.

The length of the lesion to be treated is typically visualized by introducing contrast media into the target vessel V and observing the resulting image under a fluoroscope. Radiopaque markers 272, 274, one at the distal end of the balloon 262 and one at the distal end of the outer sheath 258 may be used to visualize the length of the stent segments exposed for deployment relative to the target lesion. A number of stent segments 255 are selected to traverse the length of the lesion L as depicted in FIG. 7B.

In FIG. 7B, a lesion is identified and a 12 mm long stent is determined to be the appropriate stent size for treatment. Thus the distal-most stent segment 255, 12 mm long is selected for deployment and exposed from outer sheath 258. Outer sheath 258 is axially retracted in the proximal direction by means of a control mechanism on the proximal end of the delivery catheter 250 such as the control mechanism 656, 657 illustrated in FIGS. 6B and 6C. Once the operator has decided to deliver a 12 mm long stent, control mechanism 656 in FIG. 6B is moved into the “12” position such that pin 600 in FIG. 6C operatively couples outer sheath 258 and pusher tube 267 together in FIG. 6C (also outer sheath 658 and pusher tube 667 in FIG. 6B). Proximal retraction of slider control 657 then simultaneously retracts outer sheath 258 and pusher tube 267. As outer sheath 258 is pulled back, the selected number of stent segments 255, here one, is exposed and unconstrained from expansion. Additionally, stent valve 260 engages the remaining stent segment 254 and it is displaced proximally, creating a spacing between the stent segment selected for delivery 255, the remaining segment 254 and the distal tip of outer sheath 258. The spacing between stent segments 254 and 255 is often between 2 mm and 6 mm with the spacing between the distal tip end of outer sheath 258 and the proximal-most end of stent segment 255 having an axial length of about 1 mm to about 3 mm to permit proper balloon inflation as previously described. In FIG. 7B, one segment 255 having a length 12 mm long has been selected and exposed from outer sheath 258. Additional stent segment 254 remains covered by outer sheath 258 unless it is selected for delivery after the initial quantity exposed has been determined to be insufficient to cover the target lesion.

Referring now to FIG. 7C, the selected stent segment 255 is deployed. Expandable member 262, typically a balloon, is inflated with a fluid such as contrast media and/or saline to achieve an expanded diameter 263. Radial expansion of expandable member 262 correspondingly expands stent segments 255 against the vessel wall across lesion L. Outer sheath 258 and radiopaque marker 272 constrain inflatable member 262 and prevent deployment of the stent segment 254 remaining with the delivery catheter 250. After stent segment 255 is deployed, expanded member 263 is deflated and removed from the deployed stent segment 255, leaving stent segment 255 in a plastically deformed, expanded configuration in the vessel V, at the site of the lesion, L. Stent segment 254 remains with the delivery catheter 250 and both are removed and retracted from the vessel V.

FIGS. 7D-7F illustrate how the present embodiment can be used to deliver both stent segments 254 to form a longer prosthesis, here 18 mm long. As previously discussed and illustrated in FIG. 7A, the delivery catheter 250 is positioned at the target lesion site L. In order to expose both stent segments 254, outer sheath 258 is retracted proximally until both stent segments 254 are unconstrained from expansion. Outer sheath 258 is retracted by actuating a control mechanism such as that illustrated in FIGS. 6B and 6C. In FIG. 6B, switch 656 is set to the “18” position so that pin 600 is free to slide along slot 602 in outer sheath 658 (or outer sheath 258 in FIG. 7D). Because pin 602 is free to move, outer sheath 658 can move axially relative to pusher tube 667 the length of slot 602. Slider 657 is coupled with outer sheath 658, therefore as slider 657 is retracted proximally, outer sheath 658 also is retracted, thereby exposing stent segments 254. As outer sheath 658 retracts, stent valve or engaging member 660 slides over the proximal most stent segment 654. Stent segment 654 remains stationary because pusher tube 667 (or 267 in FIG. 7D) serves as a backstop to prevent proximal movement of the stent segment 654.

In the same step as stent segment exposure, a spacing is created between the distal end of outer sheath 258 and the proximal end of the selected group of stent segments 255, as shown in FIG. 7E. Once slider 657 in FIG. 6B reaches the end of its travel along slot 690, pin 600 engages the distal end of slot 602, thereby coupling the outer sheath 658 and pusher tube 667. As slider 657 is further withdrawn proximally along slot 692, outer sheath 658 is further withdrawn along with pusher tube 667, creating a spacing between the distal-most end of outer sheath 258 and the proximal-most end of exposed stent segments 255. This spacing typically has an axial length of about 1 mm to about 3 mm. Once the spacing is created, the balloon 262 is inflated in FIG. 7F to achieve an expanded diameter 263 which correspondingly expands stent segments 255. After the stent segments have been deployed, expanded balloon 263 may be deflated and the stent delivery catheter 250 removed from the vessel, leaving stent segments 255 implanted at the site of the lesion L.

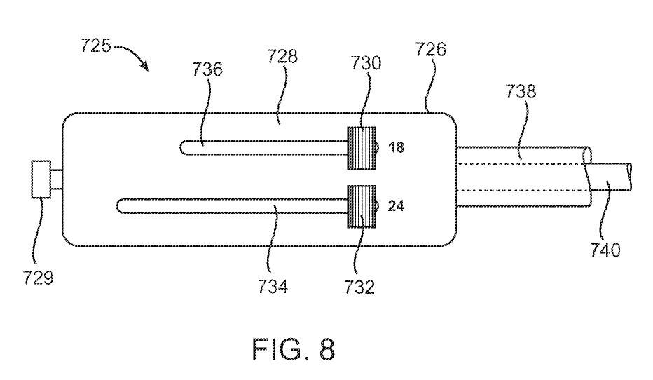

FIG. 8 illustrates an alternative handle and control mechanism that may be used in the stent delivery system of FIGS. 6A-6C and FIGS. 7A-7F. The embodiment illustrated in FIG. 8 is similar to the previous embodiment but has multiple slider controls for selecting the number of stent segments for deployment, rather than a single slider control in FIG. 6B.

Handle 726 is adapted to deliver either three or four stent segments for delivery of prostheses either 18 mm long or 24 mm long although a wide range of lengths and numbers of segments are possible. Handle 726 includes a housing 728 enclosing the components of the handle 726. The handle 726 is coupled with outer sheath 738 and is also operably coupled with pusher tube 740. Handle 726 contains two slider control mechanisms 730 and 732 disposed along handle body 728. As in the previous embodiment, the stent valve is positioned on the outer sheath 738 so as to be in engagement with the proximal-most stent when the outer sheath 738 is in its initial distal-most position.

When an 18 mm long prosthesis is to be delivered, the physician operator simply retracts the slider control mechanism 730 until it reaches the end of its travel along slot 736. The pusher tube 740 is retracted along with the outer sheath 738 in this motion to expose three stent segments, each 6 mm long. Further retraction of slider 730 retracts the outer sheath 738 with pusher tube 740 and a stent valve on the inner surface of outer sheath 738 engages and retracts the last stent segment with the outer sheath 738 and pusher tube 740. This creates a small separation between the three stent segments and the distal end of the outer sheath 738, allowing for proper balloon expansion. If a 24 mm long prosthesis is to be delivered, slider control mechanism 732 is retracted which retracts only the outer sheath 738 while the pusher tube 740 remains stationary until the stent valve clears the proximal stent segment. Further retraction moves both the outer sheath and pusher tube together thereby exposing all four stent segments and creating the appropriate spacing from the tip of the outer sheath. In both cases, whether deploying two or three stent segments, after the appropriate number of stent segments has been selected and exposed for delivery, a balloon on the distal end of the delivery system 725 may be inflated via an adapter 729 attached to handle 726. The adaptor 729 is typically a Luer connector and adaptor 729 is fluidly connected to the balloon permitting radial expansion and implantation of the stent segments into lesion L. After the stents have been deployed, the balloon is deflated and the delivery catheter is removed from the vessel.

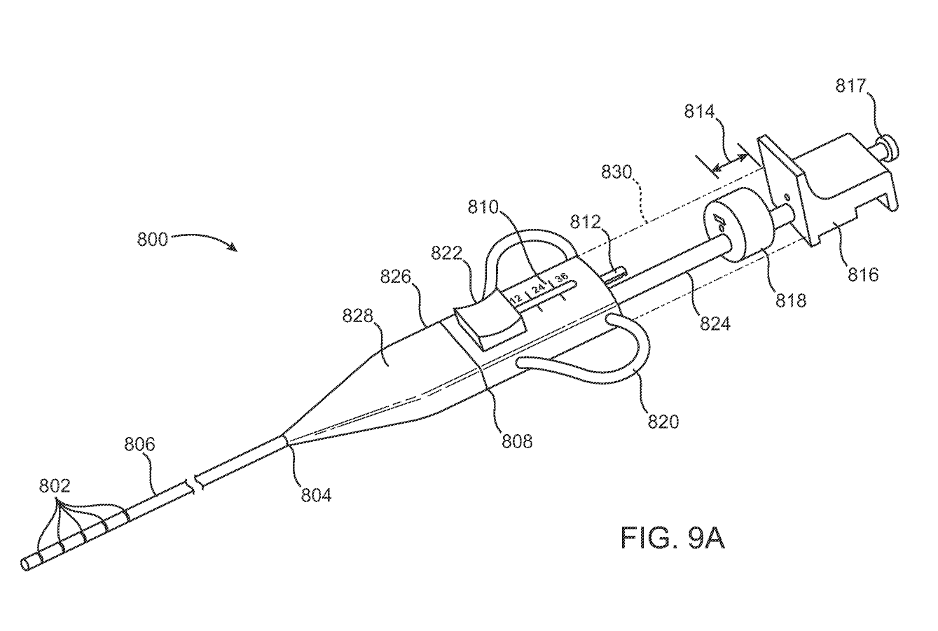

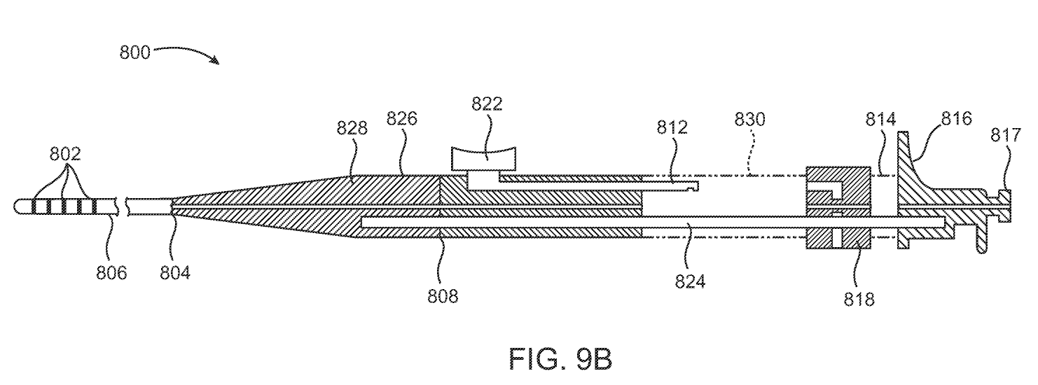

FIGS. 9A-9B illustrate another embodiment of the control mechanism in a stent delivery system. This embodiment is similar to the embodiment shown in FIGS. 3A-3B with several exceptions. Some of these differences include an alternatively shaped handle that is grasped by an operator's hand differently as well as alternative control surfaces. The embodiment in FIG. 9A shows a perspective view of stent delivery catheter 800 which is designed for a single stent deployment and allows the physician operator to deploy a prosthesis having a length ranging from 12 mm to 36 mm. In this embodiment, it is preferred that the delivery system 800 carry six, 6 mm long stent segments, although a wide range of stent segment lengths and numbers are possible.

Stent delivery system 800 comprises an outer sheath 806 connected with handle 826 on the proximal end of the delivery system 800. Strain relief 804 helps prevent kinking of the outer sheath 806. Radiopaque markers 802 on the distal end of outer sheath 806 allow a physician to estimate lesion length under fluoroscopy. The handle 826 has a housing 828 which encloses the handle components. A section of the housing 830 has been removed to allow visualization of certain internal housing components.

Handle 826 comprises a slider 822 which allows the operator to select the number of stent segments to be deployed. In this exemplary embodiment stent segments having a combined length of either 12, 18, 24, 30 or 36 mm long may be deployed. The lever 822 is slid along the calibrated slot 810 until the desired lesion length position is obtained. Detents or visual markers in the lever mechanism 822 provide tactile, visual and/or audible feedback to the user to facilitate adjustment of during operation. A displacement rod 812 is operably coupled with lever 822 such that, as lever 822 is drawn back to select the number of stent segments to be deployed, displacement rod 812 is also axially displaced a proportional distance.

Handle 826 is disposed over a rigid frame 824 which serves as a chassis for the handle components. After the number of stent segments for deployment has been selected with lever 822, lever 820 is pulled back, typically with two fingers, away from its distal-most stop position 808. Lever 820 is operably coupled with outer sheath 806, so as lever 820 slides over rigid frame 824, outer sheath 806 is also axially displaced. This motion exposes enough stent segments on the distal end of the catheter to treat the given lesion size. Lever 820 is pulled back until displacement rod 812 engages with a pusher assembly 818, also axially disposed along the rigid frame 824.

The pusher assembly 818, is operably coupled with a pusher tube and can be axially displaced in the proximal direction, reducing gap 814 to create a spacing between stent segments exposed for deployment and those remaining with the outer sheath 806. The proximal handle end 816 includes an adapter 817, often a Luer, to allow an inflation device to fluidly communicate with an expandable member on the distal end of the delivery catheter (not shown). Additionally, the proximal handle end 816 may be contoured to provide a finger or thumb rest that facilitates retraction of the activation lever 820. FIG. 9B represents a cross-sectional side view of handle 826 depicted in FIG. 9A.

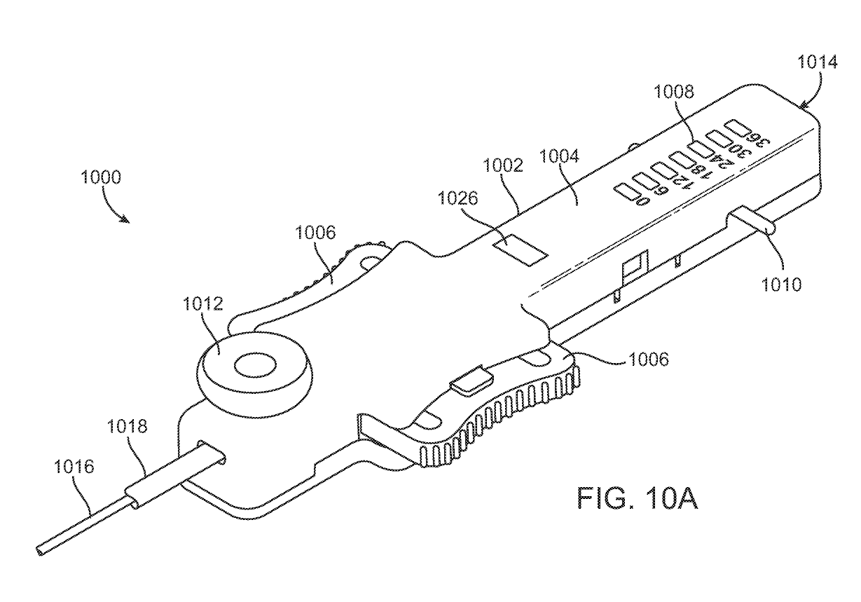

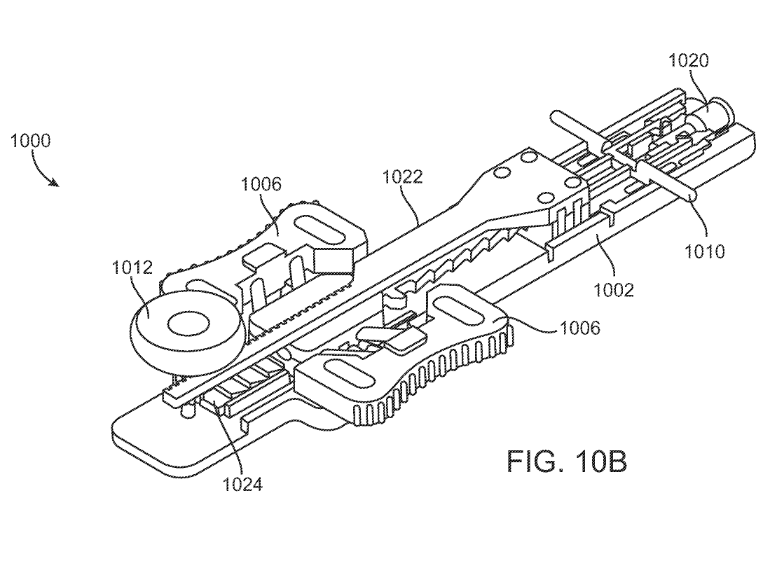

Still, yet another embodiment of a control mechanism and handle of a stent delivery system is illustrated in FIGS. 10A-10B. This embodiment differs from previous embodiments in terms of overall handle geometry as well as having a pair of triggers and rotating knob mechanism as the actuating controls. FIG. 10A shows a perspective view of the proximal end of a stent delivery system 1000, comprising a handle 1002 that is ergonomically designed and can be easily gripped by an operator. The handle 1002 comprises a housing 1004 which contains the internal components of the control mechanism used during deployment of stent segments from the delivery system. An automatic preparation set lever 1010 may be pulled back proximally to break the outer sheath 1018 free from any stent segments as a result of “stiction” that might develop during transportation and storage of the device, prior to use. A pair of triggers 1006 disposed on the side of the handle 1002 are used to select the number of stent segments for deployment. Each time the triggers 1006 are depressed, typically with a thumb and forefinger, one stent segment is exposed as outer sheath 1018 is retracted. Visual indicator 1008 allows the operator to observe how many stent segments have been selected for deployment and/or the corresponding lesion length that can be stented. In this embodiment, preferably, six, 6 mm stent segments are deployable to form a prosthesis having length ranging from 6 mm to 36 mm long.

Once the trigger actuators 1006 have been actuated and the desired number of stent segments exposed, control knob 1012 may be rotated to simultaneously retract outer sheath 1018 and pusher tube 1016 in order to create a spacing between the selected stent segments and those that remain with the delivery catheter. A stent valve (not shown) that generally takes the same form as stent valves previously described may be used to create the spacing. Indicator window 1026 provides a visual sign once an appropriate spacing has been created. This spacing typically has an axial length of about 2 mm to about 6 mm and the spacing between the distal end of outer sheath 1018 and the proximal-most end of the stent segments selected for delivery is about 1 mm to about 3 mm in order to permit proper balloon inflation and may be adjusted according to operator preference.

An inflation adaptor 1020 is disposed within the proximal end 1014 of handle 1002 during the initial phases of handle operation. The inflation adaptor 1020 generally takes the same form as other inflation adaptors discussed previously and facilitates balloon expansion. However, in this embodiment, after control knob 1012 is adjusted to create the necessary spacing for proper balloon inflation, inflation adaptor 1020 is automatically exposed at the proximal handle end 1014. This safety feature prevents premature inflation of the balloon.

FIG. 10B illustrates the handle 1002 of FIG. 10A with sections of housing 1004 removed so that some of the internal components may be observed. Outer shaft ratchet arm 1024 is operably coupled with the triggers 1006 such that as the triggers 1006 are depressed, the outer shaft ratchet arm 1024 retracts proximally, simultaneously drawing outer sheath 1018 back with it. Additionally, control knob 1012 is operably coupled with pusher actuator arm 1022 so that as knob 1012 is rotated, pusher actuator arm 1022 either moves forward or backward. This motion moves the pusher tube 1016 with it. Additionally, pusher actuator arm 1022 and outer shaft ratchet arm 1024 are operably coupled so that as pusher actuator arm 1022 is retracted proximally, so too will outer sheath 1018. This allows a spacing to be created between the stent segments selected for delivery and those remaining with the delivery catheter and the distal sheath tip. The spacing may be adjusted after stent segment delivery so that the balloon length can be adjusted for post-dilation “tacking” of the stent.

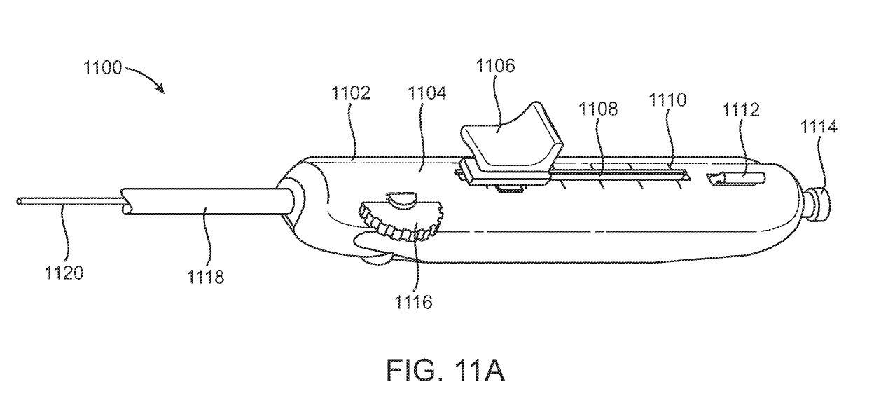

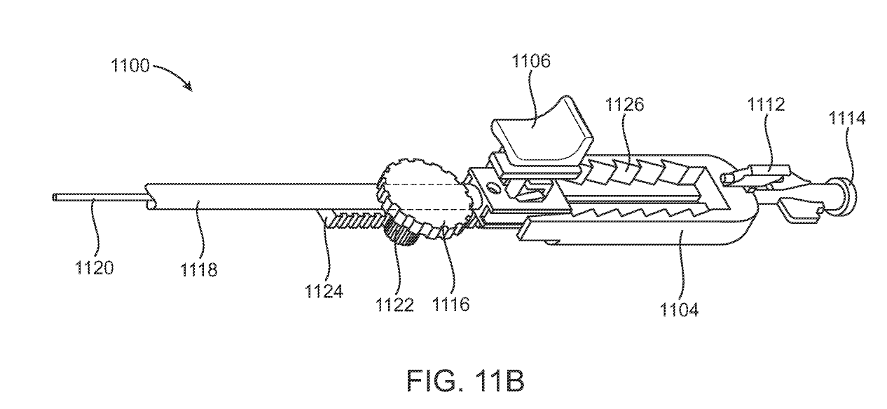

Another embodiment of a control mechanism and handle of a stent delivery system is illustrated in FIGS. 11A-11B. The embodiment in FIGS. 11A-11B differs from previously described embodiments primarily in terms of overall handle shape and the control surfaces, here a thumb wheel and thumb slide. FIG. 11A illustrates a perspective view of the proximal end of a stent delivery system 1100, comprising a handle 1102 that is ergonomically designed and can easily be gripped by an operator and operated with one hand. The handle 1102 comprises a housing 1104 which contains the internal components of the control mechanism used during deployment of stent segments from the delivery system. An adapter 1114 disposed on the proximal end of the handle 1102 allows an inflation device to be fluidly connected with the catheter and an inflatable member on the distal end of the delivery catheter (FIG. 1).

An outer sheath 1118 and pusher tube 1120 are coupled with the distal end of handle 1 102. Operator control surfaces 1106, 1112 and 1116 extend from the handle housing 1104 and permit an operator to select a number of stent segments for deployment. Slider 1106 is calibrated an allows a physician to select the number of stent segments to be deployed. This is accomplished by depressing slider 1106 downward to unlock the slider 1106 and then retracting the slider 1106 proximally along the slider slot 1108, typically with a thumb. Outer sheath 1118 is coupled with slider 1106, therefore as the slider 1106 is retracted, so too is outer sheath 1118. Retraction of outer sheath 1118 exposes stent segments on the distal end of the delivery system 1100. Detents and/or visual markers 1110 along the slider slot 1108 provide tactile, audible and/or visual feedback to facilitate the operator with stent segment selection. This is particularly useful when the physician operator is simultaneously observing a fluoroscopic viewing screen during the stenting procedure.

Once the number of stent segments to be deployed has been determined by retracting slider 1106, safety switch 1112 may be moved from a locked position to an unlocked position so as to allow separation wheel 1116 to be rotated. The safety switch 1112 prevents additional unintended use of the delivery system 1100. Separation wheel 1116 is then rotated counter-clockwise to create a spacing between the stent segments selected for deployment and those remaining with the delivery system 1100. This spacing has been previously described and is required for proper balloon inflation during stent segment deployment. The spacing is created because as separation wheel 1116 is rotated, it is coupled with pusher tube 1120 and slider 1106 and outer sheath 1118, therefore as the separation wheel is rotated, pusher tube 1120 and outer sheath 1118 are also simultaneously retracted and a stent valve (e.g. 1260 in FIG. 2A) retracts the stent segments which have not been selected for delivery.

After outer sheath 1118 has exposed stent segments to be deployed and a proper spacing has been created, an expandable member such as a balloon is inflated and the stent segments are radially expanded and implanted at the target lesion site. The expandable member is then deflated and the stent delivery catheter is removed from the lesion site. This has previously been described, for example with respect to FIGS. 2A-2E.

FIG. 11B is a perspective view of the handle 1102 with portions of the housing 1104 removed to show the internal components of the handle 1102. In addition to the components described previously with respect to FIG. 11A, the handle 1102 also includes ratchets 1126 in the handle body 1104 that calibrate slide mechanism 1106 so that each actuation or “click” of slider 1106 exposes one stent segment. Additionally, a pinion 1122 couples the separation wheel 1116 with rack 1124 so that rotation of the separation wheel 1116 will linearly translate the rack 1124 which is coupled with pusher tube 1120 and creates a spacing between stent segments selected for delivery and those remaining with the delivery catheter 1100.

After the stent segments have been expanded and implanted at the treatment site, the delivery catheter 1100 may also be used for a post-treatment dilation. This is accomplished by rotating the separation wheel 1116 in the clockwise direction to so that outer sheath 1118 is advanced distally, constraining a portion of the balloon on the distal end of the delivery catheter 1100. The balloon may then be re-inflated in order to “tack” the deployed stent segments into position.

In this embodiment, preferably six stent segments, each having a length approximately 6 mm long are deployable and can form a prosthesis having a total length ranging from about 6 mm to about 36 mm. Other stent lengths and quantities may be used in the delivery system 1100, including stent segments having different lengths.

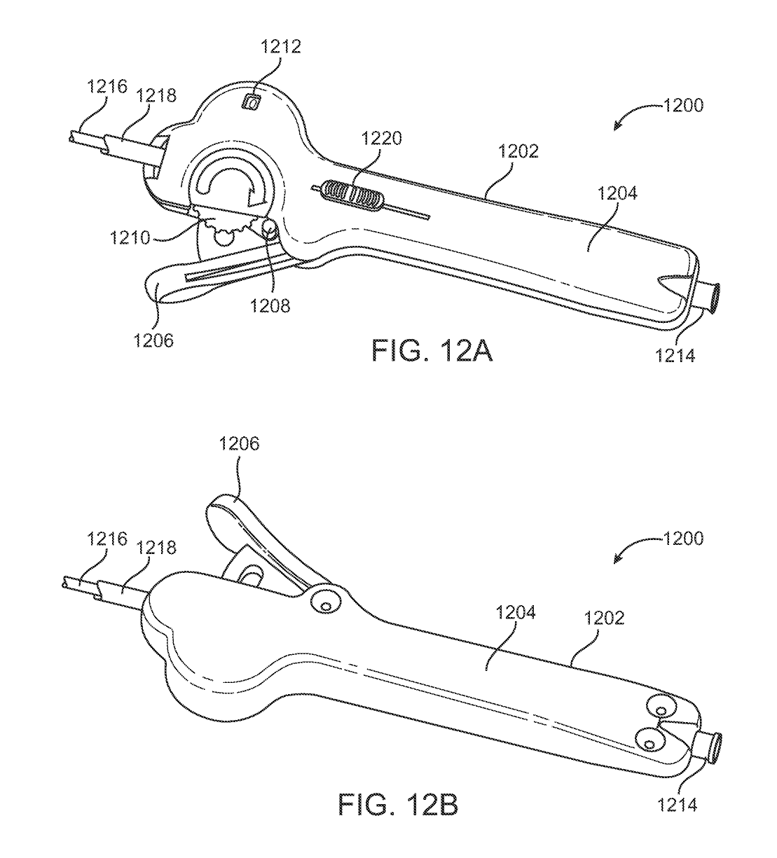

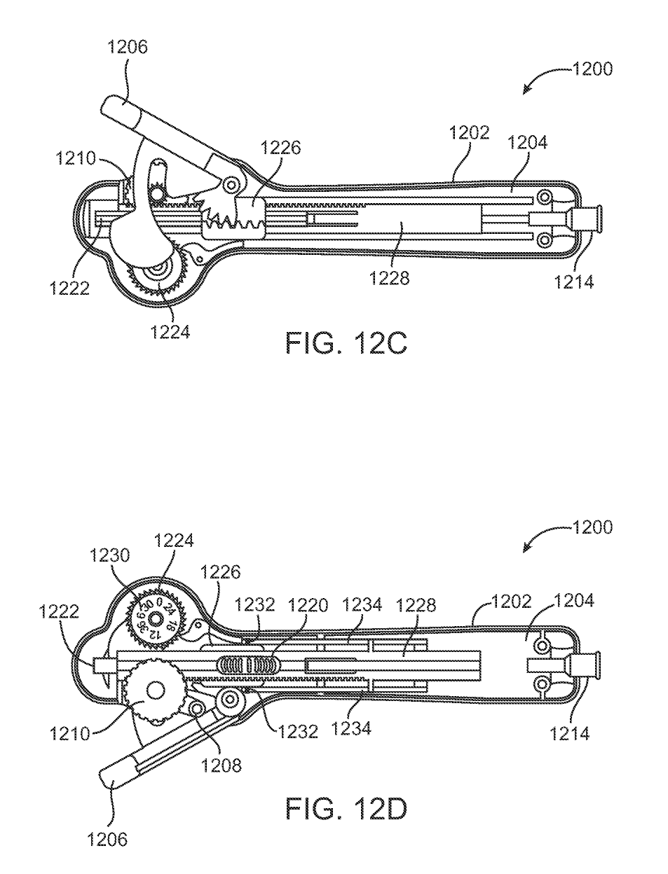

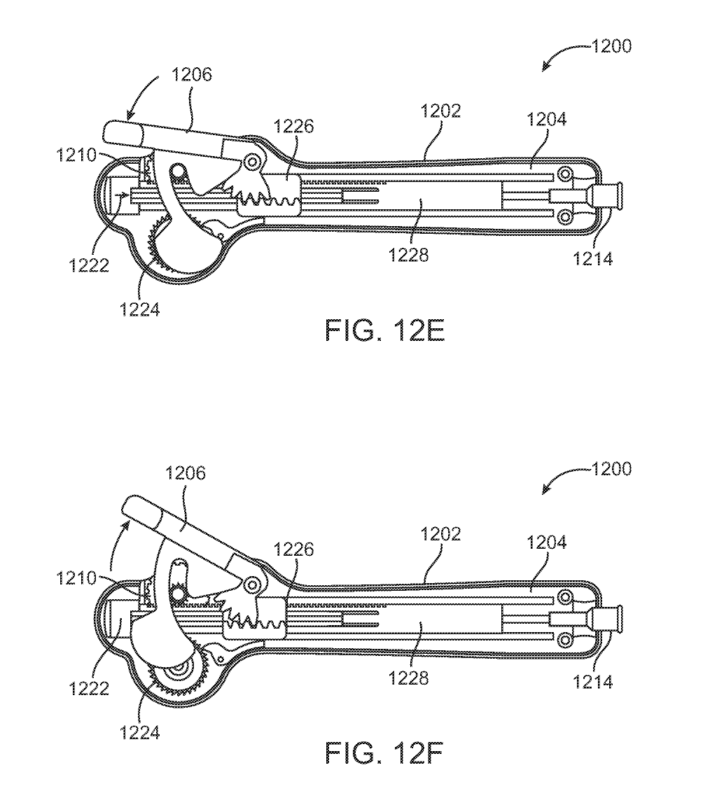

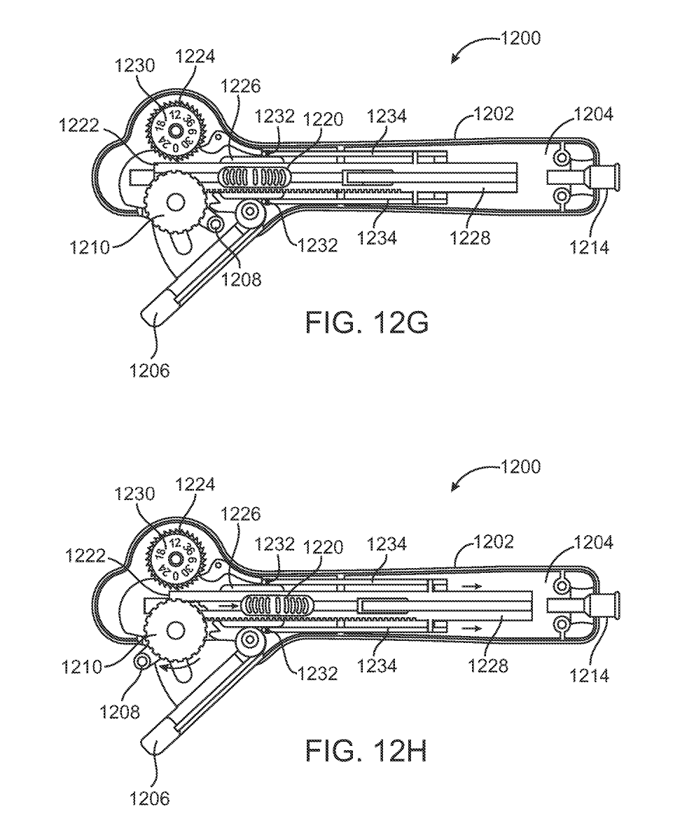

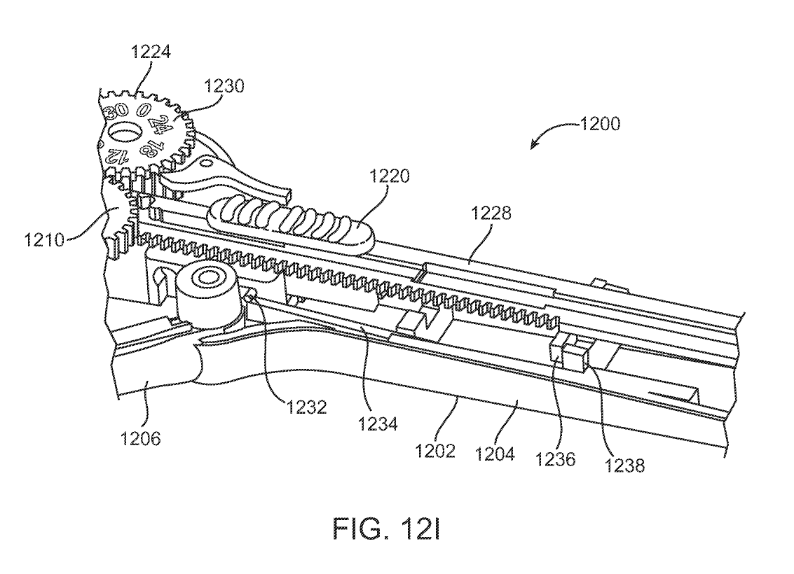

Yet another embodiment of a control mechanism and handle of a stent delivery system is illustrated in FIGS. 12A-12I. The embodiment in FIGS. 12A-12I differs from other embodiments previously described primarily in terms of handle shape and the actuation mechanisms used to expose and separate stent segments. Here, levers are used to select and separate stent segments and a rotatable wheel may be used to fine tune the spacing created between stent segments selected for delivery and those remaining with the delivery catheter.

FIG. 12A illustrates a perspective view of the proximal end of a stent delivery system 1200, comprising a handle 1202 that is ergonomically designed and can easily be gripped by an operator and operated with one hand. FIG. 12B is a bottom view of the handle 1202. The handle 1202 comprises a housing 1204 which contains the internal components of the control mechanism used during deployment of stent segments from the delivery system. An adapter 1214 disposed on the proximal end of the handle 1202 and coupled with the housing 1204 allows an inflation device to be fluidly coupled with the catheter and an inflatable member on the distal end of the delivery catheter (e.g. element 24 in FIG. 1).

An outer sheath 1218 and pusher tube 1216 are coupled with the distal end of handle 1202. Operator control surfaces 1206, 1208 and 1210 extend from the handle housing 1204 and permit an operator to select a number of stent segments for deployment. Deployment lever 1206 is actuated by a physician and allows a physician to select the number of stent segments for deployment. A single stent segment is selected after each actuation of the deployment lever through its full stroke. Outer sheath 1218 is coupled with the deployment lever 1206, therefore as the deployment lever 1206 is actuated, outer sheath 1218 is retracted proximally, thus exposing stent segments on the distal end of the delivery system 1200. A stent counter 1212 allows an operator to see how many stent segments have been selected for deployment. Each time the deployment lever 1206 is actuated and a stent segment is selected for deployment, stent counter 1212 is advanced to increase the count showing. This is useful since it allows a physician to quickly determine how many stent segments have been selected for deployment or the total prosthesis length, especially while simultaneously observing a fluoroscopic viewing screen during the stenting procedure.

Once the number of stent segments to be deployed has been selected, separation lock 1220 is moved into the unlocked position and separation lever 1208 is actuated to separate the group of stent segments selected for deployment from those remaining with the delivery catheter 1200. This spacing has been previously described and is required for proper formation of a balloon taper during balloon inflation and stent deployment. The spacing is created because separation lever 1208 is operably coupled with pusher tube 1216 and outer sheath 1218. As the separation lever 1208 is actuated, both pusher tube 1216 and outer sheath 1218 are retracted and a stent valve (e.g. 1260 in FIG. 2A) disposed on the outer sheath 1218 draws any remaining stent segments proximally away from the stent segments selected for deployment.

After outer sheath 1218 has been retracted and selected stent segments are exposed along with the necessary spacing, a fine tuning wheel 1210 coupled with the separation lever 1208 may be rotated to adjust the spacing between the group of stent segments to be deployed and those remaining with the delivery catheter 1200. An expandable member such as a balloon on the distal end of the delivery catheter 1200 is then inflated to radially expand and implant stent segments at the target lesion site. The expandable member is then deflated and the stent delivery catheter may be removed from the lesion site. This has previously been described, for example with respect to FIGS. 2A-2E. Optionally, the fine tuning wheel 1210 may be rotated to control the amount of balloon that is unconstrained by the outer sheath 1218 when post-dilation tacking of stent segments is performed.

FIG. 12C is a bottom view of handle 1202 with portions of the housing 1204 removed to show the internal components of the handle 1202. In addition to the components previously described, handle 1202 also includes outer shaft rack 1222 which is coupled to outer sheath 1218 and is actuated by separation lever 1206 via slider 1226. Lever clutch 1224 is coupled to separation lever 1206 and allows the lever 1206 to reset after it has been actuated through its full arc. Handle 1202 also includes pusher rack 1228 which is coupled with a pusher tube 1216. FIG. 12D shows the top view of handle 1202 with portions of the housing removed. From the top view, additional components such as counter wheel 1230 on clutch 1224 may be seen along with pusher rack arms 1232 and latch release 1234.

In FIG. 12E, separation lever 1206 is actuated by rotating it counter-clockwise until it reaches the end of its arc and bottoms out. This motion is translated via slider 1226 into linear motion which results in proximal retraction of outer shaft rack 1222. An outer sheath 1218 is coupled with the outer shaft rack 1222 and thus, as the outer shaft rack 1222 is retracted, so too is the outer sheath 1218 which exposes stent segments. Once separation lever 1206 has reached the end of its travel, clutch 1224 allows separation lever 1206 to re-set back to its initial position where it can be actuated again. A top view of this is seen in FIG. 12F and a bottom view is illustrated in FIG. 12G. Each actuation of separation lever 1206 exposes a stent segment and advances the stent counter 1230 seen through counter window 1212. Actuation of separation lever 1206 is repeated until the desired number of stent segments is selected.

After the number of stent segments has been selected, a spacing must be created between those stent segments selected for deployment and those remaining with the delivery catheter. The spacing is necessary for balloon taper formation during balloon inflation. In FIG. 12H, separation lever 1206 is rotated clockwise approximately 90°. Rotating the separation lever 1206 causes pusher rack 1228 to move proximally. A pusher tube 1216 is coupled with pusher rack 1228 as well as outer sheath rack 1222. Therefore, when the separation lever 1206 is rotated, both outer sheath 1218 and pusher tube 1216 are retracted proximally. A stent valve (e.g. 1260 in FIG. 2A) engages the stent segments remaining with the delivery catheter and draws them proximally, creating a spacing with the group of segments selected for delivery.

As the pusher rack 1228 is retracted proximally, pin-like arms 1232 on the pusher rack 1228 engage the latch release 1234 which disengages the pusher rack 1228 from the latch release 1234 at engagement tabs 1236, 1238. This is observed in FIG. 12I. The separation wheel 1210 may then be manually rotated counter-clockwise for fine tuning the spacing previously created. Fine tuning may be used during delivery of the stent segments or to control the amount of exposed balloon for a post-stenting dilation or tacking.

In this embodiment, preferably six stent segments, each having a length approximately 6 mm long are deployable from the delivery catheter and can form a prosthesis having a total length of approximately 36 mm. Other stent segment lengths and quantities may be used in the delivery system including stent segments having different lengths, and as in other embodiments, the stent segments may comprise a therapeutic agent. Often the therapeutic agent is an anti-restenosis agent.

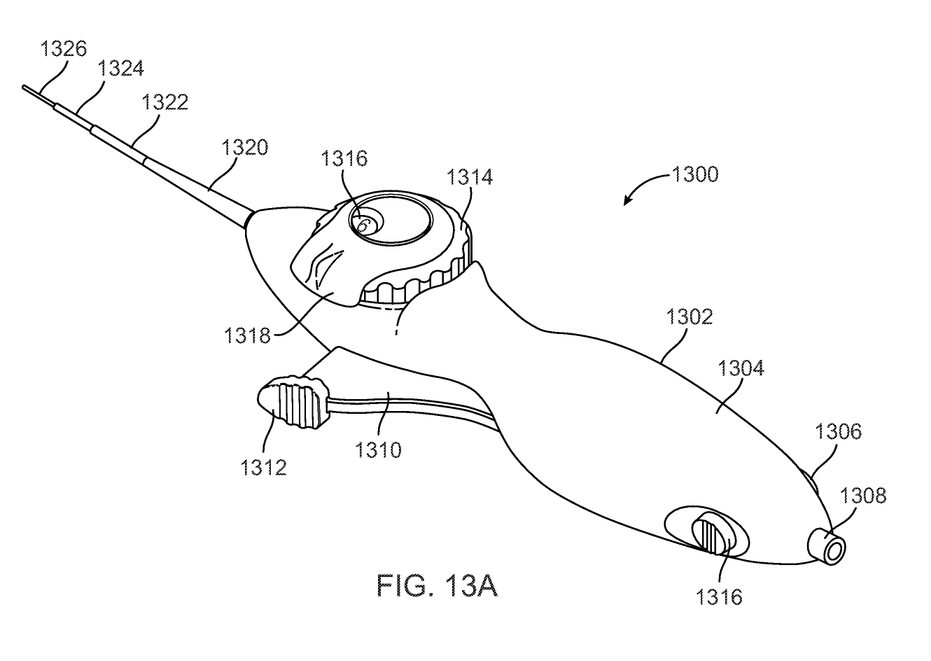

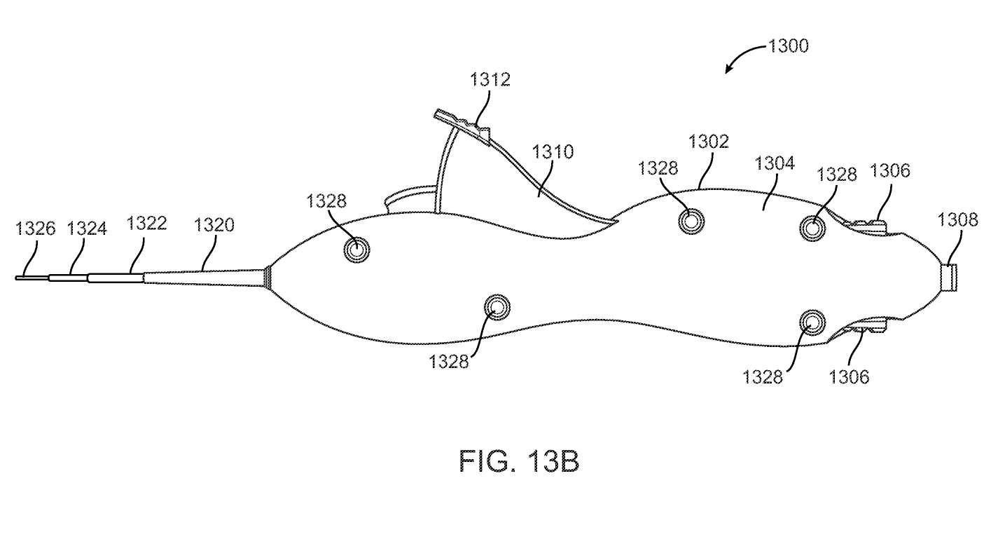

Still another embodiment of a handle and control mechanism in a stent delivery system is shown in FIGS. 13A-13B. The embodiment of FIGS. 13A-13B is similar to that described previously with respect to FIGS. 12A-12I, the major difference being the overall shape of the handle and the control mechanism surfaces. The internal mechanism of this embodiment operates in the same manner as previously described for FIGS. 12A-12I.

FIG. 13A illustrates a perspective view of the proximal end of a stent delivery system 1300, comprising a handle 1302 that is ergonomically designed and can easily be gripped by an operator. The outer surface of handle 1302 is contoured to fit comfortably in the palm of an operator's hand thereby allowing the operator to manipulate control surfaces with a thumb and forefinger. FIG. 13B is a bottom view of the handle 1302. Handle 1302 comprises a housing 1304 which contains the internal components of the control mechanism used during deployment of stent segments from the delivery system. An adapter 1308 is disposed on the proximal end of handle 1202 and is coupled with the housing 1304. Adaptor 1308 allows an inflation device to be fluidly coupled with the catheter and an inflatable member on the distal end of the delivery catheter (for example element 24 in FIG. 1). A release mechanism, here a pair of squeezable buttons 1306 is coupled with inner shaft 1326. Buttons 1306 are squeezed inwardly and then retracted in order to draw catheter inner shaft 1326 slightly proximally, thereby releasing the catheter shaft 1326 from sticking to pusher 1324 and outer sheath 1322 which may be acquired during transportation and storage of the delivery catheter. This has been previously discussed above in reference to auto prep set lever 1010 in FIG. 10A.

Outer sheath 1322, pusher tube 1324 and inner catheter shaft 1326 are coupled with the distal end of handle 1302. A strain relief 1320 is disposed over a portion of outer sheath 1322 adjacent to where outer sheath 1322 joins handle 1302 and helps prevent kinking of the outer sheath 1322 during use. Operator control surfaces include a deployment lever 1310 extending from handle housing 1304 and having a surface 1312 which is actuated by an operator's thumb or other finger. Actuating the deployment lever 1310 allows a physician to select the number of stent segments for deployment. A single stent segment is selected after each actuation of deployment lever 1310 through its full stroke. Outer sheath 1322 is coupled with the deployment lever 1310, therefore as deployment lever 1310 is actuated, outer sheath 1322 is retracted proximally, thereby exposing stent segments on the distal end of the delivery catheter 1300. A stent counter 1316 allows an operator to see how many stent segments have been selected for deployment. Each time the deployment lever 1310 is actuated and a stent segment is selected for deployment, stent counter 1316 is advanced and increases the count showing. This is advantageous since it allows a physician to quickly see how many stent segments have been selected for deployment or the total prosthesis length, especially while simultaneously observing a fluoroscopic viewing screen during the stenting procedure.

Once the number of stent segments to be deployed has been selected, separation knob 1314 may be actuated by rotation to separate the group of stent segments selected for deployment from those remaining with the delivery catheter 1300. This spacing has been previously described and is necessary for proper formation of a balloon taper during inflation of the balloon and stent deployment. The spacing is created because separation knob 1314 is operably coupled with pusher tube 1324 and outer sheath 1322. As the separation knob 1314 is actuated, both pusher tube 1324 and outer sheath 1322 are retracted and a stent valve or engaging member (e.g. 1260 in FIG. 2A) disposed on outer sheath 1322 draws any remaining stent segments proximally away from those selected for deployment.

After outer sheath 1322 has been retracted and selected stent segments are exposed along with the necessary spacing, separation knob 1314 may be further rotated to either increase or decrease the spacing between the group of stent segments selected for deployment and those remaining with the delivery catheter 1300. An expandable member such as a balloon on the distal end of delivery catheter 1300 may then be inflated to radially expand and implant the stent segments at the target lesion site. The expandable member is then deflated and the stent delivery catheter 1300 may be removed from the lesion site. This has been discussed above, for example with respect to FIGS. 2A-2E. Additionally, selection lever 1318 may also be actuated so that rotation of actuation knob 1314 decouples motion of the pusher 1324 from motion of the outer sheath 1322. Thus, further actuation of knob 1314 controls the amount of balloon that is unconstrained by the outer sheath 1322 when post-dilation tacking of stent segments is performed. FIG. 13B is a bottom view of handle 1302 showing many of the same components as in FIG. 13A. Handle 1302 is composed of a top half and a bottom half. The top and bottom portions of handle 1302 may be joined together using screws 1328 as well as by adhesively bonding the two halves together or ultrasonically welding. The internal mechanisms of handle 1302 are the same as the internal mechanisms of FIGS. 12A-12I and have been previously discussed.

In this embodiment, preferably six stent segments, each having a length approximately 6 mm long are deployable from the delivery catheter and can form a prosthesis having a total length of approximately 36 mm. Other stent segment lengths and quantities may be used in the delivery system including stent segments having different lengths, and as in other embodiments, the stent segments may comprise a therapeutic agent. Often the therapeutic agent is an anti-restenosis agent.

Other mechanical mechanisms are well known to those skilled in the art, such as but not limited to ratchets, rotating knobs, rack and pinions, etc. Many of these can easily be employed to achieve the same results as described above and thus the embodiments described should not be limited to solely the mechanisms described herein. Although the foregoing invention has been described in some detail by way of illustration and example, for purposes of clarity of understanding, it will be obvious that various alternatives, modifications and equivalents may be used and the above description should not be taken as limiting in scope of the invention which is defined by the claims.

What is claimed is:

1. An apparatus for delivering prostheses in a body lumen, the apparatus comprising:
an elongated flexible member having a proximal end and a distal end;
a plurality of radially-expandable prostheses in an unexpanded condition arranged axially along the elongated flexible member near the distal end;
an outer sheath slidably disposed over at least a portion of the prostheses; and
a control mechanism coupled with the outer sheath and the elongated flexible member wherein the control mechanism includes means for selecting a plurality of prostheses to deploy in a range from 2 to 20, and wherein the control mechanism retracts the sheath a fixed distance to expose the selected plurality of prostheses outside the sheath in the unexpanded condition.

2. An apparatus as in claim 1, wherein the control mechanism is further adapted to create a space free of the prostheses between a proximal prosthesis in the selected plurality and a distal end of the sheath with the prostheses in the unexpanded condition.

3. An apparatus as in claim 2, wherein the control mechanism creates the space with a predetermined length.

4. An apparatus as in claim 3, wherein the control mechanism is adapted to permit operator adjustment of the length of the space after creating the space with the predetermined length.

5. An apparatus as in claim 4, wherein the control mechanism is adapted to be held by an operator using one hand while simultaneously being actuated with one or more fingers or a thumb of the one hand.

6. An apparatus as in claim 1, further comprising a handle coupled to the proximal end of the elongated flexible member.

7. An apparatus as in claim 1, wherein actuation of the control mechanism retracts the sheath a fixed distance equal to a predetermined multiple of the length of each prosthesis.

8. An apparatus as in claim 6, wherein the control mechanism comprises an actuator movably coupled to the handle.

9. An apparatus as in claim 8, wherein the handle is adapted to be held by an operator using one hand while simultaneously actuating the actuator with one or more fingers or a thumb of the one hand.

10. An apparatus as in claim 6, wherein the control mechanism comprises an axially movable slider disposed on the handle.

11. An apparatus as in claim 6, wherein the control mechanism comprises a pair of triggers pivotably coupled to the handle.

12. An apparatus as in claim 6, wherein the control mechanism comprises a lever.

13. An apparatus as in claim 8, wherein the actuator is movable through a fixed distance, and each actuation of the actuator through the fixed distance retracts the outer sheath a distance equal to the length of one of the prostheses or a predetermined multiple thereof.

14. An apparatus as in claim 2, wherein the control mechanism comprises an actuator movable through a fixed distance, and each actuation of the actuator through the fixed distance retracts the outer sheath a distance equal to the length of one prosthetic segment plus the predetermined length.

15. An apparatus as in claim 6, wherein the control mechanism comprises a selector switch disposed on the handle, the selector switch having a plurality of positions corresponding to different numbers of the prostheses to be included in the selected plurality.

16. An apparatus as in claim 15, wherein the control mechanism further comprises a movable actuator, wherein movement of the actuator retracts the sheath a distance which varies according to the position of the selector switch.

17. An apparatus as in claim 16, wherein the actuator is movable through a distance, wherein the distance varies according to the position of the selector switch.

18. An apparatus as in claim 16, wherein the actuator is movable through a fixed distance, and wherein moving the actuator through the fixed distance retracts the sheath a distance equal to the length of one of the prostheses or a predetermined multiple thereof.

19. An apparatus as in claim 6, wherein the control mechanism comprises a ratchet mechanism.

20. An apparatus as in claim 6, wherein the control mechanism comprises a rotationally movable mechanism.

21. An apparatus as in claim 6, wherein the control mechanism comprises a linearly movable mechanism.

22. An apparatus as in claim 21, wherein the control mechanism comprises a plurality of axially movable sliders on the handle, the sliders being movable through a fixed distance.

23. An apparatus as in claim 22, wherein a first of the sliders is movable through a first fixed distance and a second of the sliders is movable through a second fixed distance different than the first distance.

24. An apparatus as in claim 23, wherein when the first slider is moved through the first fixed distance the selected plurality of prostheses exposed outside the sheath is a different number than when the second slider is moved through the second fixed distance.

25. An apparatus as in claim 6, wherein the control mechanism is adapted to provide audible, visual or tactile feedback during actuation of the control mechanism to indicate the number of prostheses in the selected plurality.

26. An apparatus as in claim 6, wherein the control mechanism comprises a first control adapted to expose the selected number of prostheses and a second control adapted to create the spacing.

27. An apparatus as in claim 26, wherein the second control is an actuator movably coupled to the handle.

28. An apparatus as in claim 27, wherein the handle is adapted to be held by an operator using one hand while simultaneously actuating the second control with one or more fingers or a thumb of the one hand.

29. An apparatus as in claim 26, wherein the second control comprises an axially movable slider disposed on the handle.

30. An apparatus as in claim 26, wherein the second control comprises a lever pivotably coupled to a handle.

31. An apparatus as in claim 26, wherein the second control is rotationally movable.

32. An apparatus as in claim 26, wherein the second control is linearly movable.

33. An apparatus as in claim 26, wherein the second control is movable through a predetermined distance and wherein moving the second control through the fixed distance creates the spacing with a predetermined length.

34. An apparatus as in claim 33, wherein the second control is not movable beyond the predetermined distance.

35. An apparatus as in claim 33, wherein the second control provides feedback to the operator when moved through the predetermined distance.

36. An apparatus as in claim 35, wherein the feedback comprises a visual indicator associated with the second control.

37. An apparatus as in claim 35, wherein the feedback comprises a detent engaged by the second control.

38. An apparatus as in claim 33, wherein after moving through the fixed distance the second control is further movable to adjust the length of the spacing.

39. An apparatus as in claim 1, further comprising a pusher element disposed on the elongated flexible member and adapted to prevent the prostheses from axially moving toward the proximal end of the elongated flexible member.

40. An apparatus as in claim 1, further comprising an engaging member disposed near a distal end of the outer sheath and adapted to engage one or more of the prostheses so as to facilitate movement of the prostheses axially on the elongated flexible member.

41. An apparatus as in claim 1, further comprising a balloon coupled to the flexible member, wherein the prosthetic segments are positionable on the balloon for expansion therewith.

42. An apparatus as in claim 1, wherein the prostheses are self-expanding.

43. An apparatus as in claim 1, further comprising an expandable member near the distal end of the elongated flexible member.

44. An apparatus as in claim 43, wherein the sheath is adapted to constrain the expandable member from expansion when positioned over the expandable member.

45. An apparatus as in claim 44, wherein upon retraction of the sheath a selected portion of the expandable member is exposed for expansion of the selected plurality of prostheses.

46. An apparatus as in claim 43, wherein the expandable member is a balloon.

47. An apparatus as in claim 1, wherein the plurality of prostheses carry a therapeutic agent adapted to be released therefrom.

48. An apparatus as in claim 47, wherein the therapeutic agent comprises an anti-restenosis agent.

49. An apparatus as in claim 1, wherein the plurality of prostheses have a length in the range from about 2 mm to about 20 mm.

50. An apparatus as in claim 1, wherein the plurality of prostheses comprise segments having a length about 3 mm to 10 mm.

51. An apparatus as in claim 1, wherein the plurality of prostheses have the same length.

52. An apparatus as in claim 1, further comprising a switch having a first position and a second position, wherein upon actuation the control mechanism exposes the prostheses when the switch is in the first position and creates the spacing when the switch is in the second position.

53. An apparatus as in claim 1, wherein the plurality of prostheses have two or more lengths.

54. An apparatus as in claim 1, wherein the plurality of prostheses have ends in engagement with one another prior to deployment.

55. An apparatus as in claim 1, further comprising an indicator adapted to display the selected number of prostheses exposed from the sheath.

56. An apparatus as in claim 1, wherein the control mechanism comprises a length selector having a plurality of settings, each setting corresponding to a number of prostheses to be exposed, whereby upon actuation the sheath is retracted a distance to expose the number of prostheses corresponding to a selected setting.

57. An apparatus for delivering prostheses in a body lumen, the apparatus comprising:
an elongated flexible member having a proximal end and a distal end;
a plurality of prostheses arranged axially along the elongated flexible member near the distal end;
an outer sheath slidably disposed over at least a portion of the prostheses; and
a control mechanism coupled with the outer sheath and the elongated flexible member, the control mechanism having a first actuator which retracts the outer sheath a distance for exposing a selected number of prostheses, the control mechanism also having a second actuator which creates a space free of the prostheses between a proximal prosthesis in the selected number of prostheses and a distal end of outer sheath.

58. An apparatus as in claim 57, wherein the control mechanism creates the space with a predetermined length.

59. An apparatus as in claim 58, wherein the control mechanism is adapted to permit operator adjustment of the length of the space after creating the space with the predetermined length.

60. An apparatus as in claim 57, wherein actuation of the control mechanism retracts the sheath a fixed distance equal to a predetermined multiple of the length of each prosthesis.

61. An apparatus as in claim 57, wherein the first actuator is movable through a fixed distance, and each actuation of the first actuator through the fixed distance retracts the outer sheath a distance equal to the length of one prosthetic segment or a predetermined multiple thereof.

62. An apparatus as in claim 57, wherein the control mechanism comprises a plurality of axially movable sliders, the sliders being movable through a fixed distance.

63. An apparatus as in claim 62, wherein a first of the sliders is movable through a first fixed distance and a second of the sliders is movable through a second fixed distance different than the first fixed distance.

64. An apparatus as in claim 63, wherein the first slider is moved through the first fixed distance the selected number of prostheses exposed outside the sheath is a different number than when the second slider is moved through the second fixed distance.

65. An apparatus as in claim 57, further comprising a handle coupled to the proximal end of the elongated flexible member.

66. An apparatus as in claim 65, wherein the handle is adapted to be held by an operator using one hand while simultaneously actuating the first actuator or the second actuator with the fingers or thumb of the one hand.

67. An apparatus as in claim 57, wherein the first actuator comprises an axially movable slider.

68. An apparatus as in claim 57, wherein the first actuator comprises a pair of triggers.

69. An apparatus as in claim 57, wherein the first actuator comprises a lever.

70. An apparatus as in claim 57, wherein the first actuator comprises a selectable switch.

71. An apparatus as in claim 57, wherein the first actuator comprises a ratchet mechanism.

72. An apparatus as in claim 57, wherein the first actuator comprises a rotationally movable mechanism.

73. An apparatus as in claim 57, wherein the first actuator comprises a linearly movable mechanism.

74. An apparatus as in claim 57, wherein the first actuator is adapted to provide audible, visual or tactile feedback during actuation of the first actuator, to indicate the number of prostheses in the selected number.

75. An apparatus as in claim 57, wherein each actuation of the first actuator retracts the outer sheath a distance equal to the length of one of the prostheses.

76. An apparatus as in claim 57, wherein the second actuator comprises an axially movable slider.

77. An apparatus as in claim 57, wherein the second actuator comprises a lever.

78. An apparatus as in claim 57, wherein the second actuator comprises a rotationally movable mechanism.

79. An apparatus as in claim 57, wherein the second actuator comprises a linearly movable mechanism.

80. An apparatus as in claim 57, wherein the second actuator is movable through a predetermined distance and wherein moving the second actuator through the predetermined distance creates the spacing with a predetermined length.

81. An apparatus as in claim 80, wherein the second actuator is not movable beyond the predetermined distance.

82. An apparatus as in claim 80, wherein the second control provides feedback to the operator when moved through the predetermined distance.

83. An apparatus as in claim 82, wherein the feedback comprises a visual indicator associated with the second actuator.

84. An apparatus as in claim 82, wherein the feedback comprises a detent engaged by the second actuator.

85. An apparatus as in claim 80, wherein after moving through the fixed distance the second actuator is further movable to adjust the length of the spacing.

86. An apparatus as in claim 57, further comprising a pusher element disposed on the elongated flexible member and adapted to prevent the prostheses from axially moving toward the proximal end of the elongated flexible member.

87. An apparatus as in claim 57, further comprising an engaging member disposed near a distal end of the outer sheath and adapted to engage one or more of the prostheses so as to facilitate movement of the prostheses axially on the elongated flexible member.

88. An apparatus as in claim 57, wherein the prostheses are balloon expandable.

89. An apparatus as in claim 57, wherein the prostheses segments are self-expanding.

90. An apparatus as in claim 57, further comprising an expandable member coupled to the flexible member, wherein the prostheses are positionable on the expandable member for expansion therewith.

91. An apparatus as in claim 90, wherein the expandable member is a balloon.

92. An apparatus as in claim 90, wherein the sheath is adapted to constrain the expandable member from expansion when positioned over the expandable member.

93. An apparatus as in claim 92, wherein upon retraction of the sheath a selected portion of the expandable member is exposed for expansion of the selected number of prostheses.

94. An apparatus as in claim 57, wherein the plurality of prostheses carry a therapeutic agent adapted to be released therefrom.

95. An apparatus as in claim 94, wherein the therapeutic agent comprises an anti-restenosis agent.

96. An apparatus as in claim 57, wherein the plurality of prostheses have a length in the range from about 2 mm to about 20 mm.

97. An apparatus as in claim 57, wherein the plurality of prostheses have a length about 3 mm to about 10 mm.

98. An apparatus as in claim 57, wherein the plurality of prostheses have the same length.

99. An apparatus as in claim 57, wherein the plurality of prostheses have two or more lengths.

100. An apparatus as in claim 57, wherein the plurality of prostheses have ends in engagement with each other prior to deployment.

101. An apparatus as in claim 57, further comprising an indicator adapted to display the selected number of prostheses.

102. An apparatus for delivering a prosthesis in a body lumen, the apparatus comprising:
an elongated flexible member having a proximal end and a distal end;
at least a first and a second radially expandable prosthesis arranged axially along the elongated flexible member, near the distal end, the first and second prosthetic segments being in an unexpanded condition;
an outer sheath slidably disposed over at least a portion of the prostheses; and
a control mechanism coupled to the outer sheath and the elongated flexible member, wherein the control mechanism has at least two settings, wherein upon actuation of the control mechanism in the first setting the outer sheath is retracted to expose only the first prosthesis in the unexpanded condition and in the second setting the outer sheath is retracted to expose both the first and the second prostheses in the unexpanded condition.

103. An apparatus as in claim 102, wherein upon actuation of the control mechanism in the first setting the outer sheath is retracted to expose only the first prosthesis and create a preselected spacing between the first prosthesis and a distal end of the outer sheath.

104. An apparatus as in claim 103, wherein upon actuation of the control mechanism in the second setting the outer sheath is retracted to expose both the first and second prostheses and create the preselected spacing between the second prosthesis and a distal end of the outer sheath.

105. An apparatus as in claim 102, further comprising a handle adjacent to the proximal end of the elongated flexible member.

106. An apparatus as in claim 105, wherein the handle is adapted to be held by an operator using one hand while simultaneously actuating the control mechanism with the fingers or thumb of the one hand.

107. An apparatus as in claim 102, wherein the control mechanism comprises a sliding switch selectable between the two settings.

108. An apparatus as in claim 102, wherein the control mechanism comprises an axially movable actuator.

109. An apparatus as in claim 102, wherein the control mechanism comprises two axially movable sliders.

110. An apparatus as in claim 102, wherein the at least first and second prostheses are balloon expandable.

111. An apparatus as in claim 102, wherein the at least first and second prostheses are self-expanding.

112. An apparatus as in claim 102, further comprising an expandable member near the distal end of the elongated flexible member.

113. An apparatus as in claim 112, wherein the expandable member is a balloon.

114. An apparatus as in claim 102, wherein the at least first and second prostheses carry a therapeutic agent adapted to be released therefrom.

115. An apparatus as in claim 114, wherein the therapeutic agent comprises an anti-restenosis agent.

116. An apparatus as in claim 102, wherein the at least first and second prostheses have a length in the range from about 2 mm to about 20 mm

117. An apparatus as in claim 102, wherein the at least first and second prostheses have different lengths.

118. An apparatus as in claim 102, wherein the at least first and second prostheses have ends in engagement with each other prior to deployment.

119. An apparatus as in claim 102, further comprising a pusher element disposed on the elongated flexible member and adapted to prevent the at least first and second prostheses from axially moving toward the proximal end of the elongated flexible member.

120. An apparatus as in claim 102, further comprising an engaging member disposed near a distal end of the outer sheath and adapted to engage one or more of the prostheses so as to facilitate movement of the prostheses axially on the elongated flexible member.

121. An apparatus as in claim 120, wherein the engaging member is disposed a distance from a distal end of the sheath selected so that the engaging member engages only the second prosthesis when the sheath is positioned over both of the prostheses.

122. An apparatus as in claim 121, wherein the distance is greater than the length of the first prosthesis.

123. An apparatus as in claim 102, wherein the control mechanism is operatively coupled with the outer sheath.

124. An apparatus as in claim 102, wherein the control mechanism is operatively coupled with the pusher element.

125. An apparatus as in claim 102, wherein the pusher element is operatively coupled with the outer sheath.

126. An apparatus as in claim 125, wherein the control mechanism comprises an actuator for selectively coupling the pusher element to the outer sheath, the actuator having a first position in which the pusher element is retractable with the sheath, and a second position in which the pusher element remains stationary as the sheath is retracted.

127. An apparatus as in claim 125, wherein the pusher element comprises a pin slidably disposed in a slot in the outer sheath

128. A method for delivering prostheses to a body lumen, the method comprising:
positioning an elongated flexible member at a first treatment site having a first lesion length, the elongated flexible member having a plurality of prostheses axially arranged thereon and covered by an outer sheath;
selecting a length setting from a plurality of length settings on a control mechanism disposed on the elongated flexible member, the length setting corresponding to a first group of prostheses for deployment;
actuating the control mechanism disposed on the elongated flexible member so as to retract the outer sheath a predetermined distance based on the length setting so that the first group of prostheses are unconstrained from expansion;
separating the first group of prostheses from any remaining prostheses; and
deploying the first group of prostheses at the site of the lesion.

129. A method as in claim 128, wherein selecting a length comprises linearly moving an actuator.

130. A method as in claim 128, wherein selecting a length comprises rotationally moving an actuator.

131. A method as in claim 128, wherein selecting a length comprises actuating a pair of triggers pivotably coupled to a handle.

132. A method as in claim 128, wherein selecting a length comprises actuating a lever.

133. A method as in claim 128, wherein selecting a length comprises actuating a switch.

134. A method as in claim 128, wherein deploying the first group comprises radially expanding an expandable member disposed on the elongate flexible member.

135. A method as in claim 134, wherein the expandable member is a balloon.

136. A method as in claim 128, wherein the plurality of prostheses carry a therapeutic agent adapted to be released therefrom.

137. A method as in claim 136, wherein the therapeutic agent comprises an anti-restenosis agent.

138. A method as in claim 128, wherein the plurality of prostheses have ends in engagement with each other prior to deployment.

139. A method as in claim 128, wherein actuating the control mechanism comprises actuating the control mechanism with a thumb or finger.

140. A method as in claim 128, wherein separating the first group comprises actuating the control mechanism so as to create a space free of the prostheses between a proximal prosthesis in the first group and a distal end of the outer sheath with the prostheses in the unexpanded condition.

141. A method as in claim 140, wherein actuating the control mechanism creates the space with a predetermined length.

142. A method as in claim 141, wherein separating the first group further comprises adjusting the length of the space by actuating the control mechanism after creating the space with the predetermined length.

143. A method as in claim 128, wherein actuating the control mechanism comprises moving the actuator through a fixed distance thereby retracting the outer sheath a distance equal to the length of one prosthesis or a predetermined multiple thereof.

144. A method as in claim 141, wherein actuating the control mechanism comprises moving the actuator through a fixed distance thereby retracting the outer sheath a distance equal to the length of one prosthesis or a predetermined multiple thereof plus the space.

145. A method as in claim 128, wherein selecting a length setting comprises adjusting the position of a selector switch having a plurality of positions corresponding to different numbers of the prostheses to be included in the first group.

146. A method as in claim 128, wherein selecting a length comprises adjusting at least one of a plurality of axially movable sliders, the sliders being movable through a fixed distance.

147. A method as in claim 146, wherein a first of the sliders is movable through a first fixed distance and a second of the sliders is movable through a second fixed distance different than the first distance.

148. A method as in claim 147, wherein moving the first slider through the first fixed distance exposes a number of prostheses outside the sheath different than when moving the slider through the second fixed distance.

149. A method as in claim 128, wherein selecting a length setting comprises providing feedback to an operator, the feedback indicating the selected length.

150. A method as in claim 149, wherein the feedback comprises a visual indicator.

151. A method as in claim 149, wherein the feedback comprises a detent engaged by the control mechanism.

152. A method as in claim 128, wherein actuating the control mechanism further comprises preventing the prostheses from axially moving toward the proximal end of the elongated flexible member.

153. A method for delivering prostheses to a body lumen, the method comprising:
positioning an elongated flexible member at a first treatment site having a first lesion length, the elongated flexible member having at least a first and a second prosthesis axially arranged thereon and covered by an outer sheath;
selecting a length setting from at least two length settings on a control mechanism disposed on the elongated flexible member, the length setting corresponding to a number of prostheses for deployment;
actuating the control mechanism wherein actuating the control mechanism in the first setting retracts the outer sheath to expose only the first prosthesis and create a pre-selected spacing between the first prosthesis and the second prosthesis, and wherein actuating the control mechanism in the second setting retracts the outer sheath to expose both the first and the second prostheses and create a pre-selected spacing between the second prosthesis and a distal end of the outer sheath; and
deploying the number of prostheses at the site of the lesion.

154. A method as in claim 153, wherein actuating the control mechanism comprises actuating the control mechanism with a thumb or finger.

155. A method as in claim 153, wherein selecting a length setting comprises adjusting the position of a selector switch having a plurality of positions corresponding to different numbers of the prostheses to be included in the first group.

156. A method as in claim 153, wherein selecting a length comprises adjusting at least one of a plurality of axially movable sliders, the sliders being movable through a fixed distance.

157. A method as in claim 153, wherein selecting a length setting comprises providing feedback to an operator, the feedback indicating the selected length.

158. A method as in claim 157, wherein the feedback comprises a visual indicator.

159. A method as in claim 157, wherein the feedback comprises a detent engaged by the control mechanism.

160. A method as in claim 153, wherein actuating the control mechanism further comprises preventing the prostheses from axially moving toward the proximal end of the elongated flexible member.

161. A method as in claim 153, wherein actuating the control mechanism comprises linearly moving the control mechanism.

162. A method as in claim 153, wherein selecting a length comprises linearly moving an actuator.

163. A method as in claim 153, wherein selecting a length comprises actuating a switch.

164. A method as in claim 153, wherein selecting a length comprises moving an actuator with a thumb.

165. A method as in claim 153, wherein selecting a length comprises moving an actuator with a finger or thumb.

166. A method as in claim 153, wherein actuating the control mechanism comprises moving the control mechanism with a finger or thumb.

167. A method as in claim 153, wherein deploying the number of prostheses comprises radially expanding an expandable member disposed on the elongate flexible member.

168. A method as in claim 167, wherein the expandable member is a balloon.

169. A method as in claim 153, wherein the at least first and second prostheses carry a therapeutic agent adapted to be released therefrom.

170. A method as in claim 169, wherein the therapeutic agent comprises an anti-restenosis agent.

171. A method as in claim 153, wherein the at least first and second prostheses have ends in engagement with each other prior to deployment.
